(19) United States
(12) Patent Application Publication
(10) Pub. No.: US 2026/0308517 A1
(43) Pub. Date: Oct. 8, 2026

(54) ENGINEERED CELLS WITH ENHANCED TEAD PROTEIN TRANSCRIPTIONAL ACTIVITIES AND METHODS OF USE THEREOF

(71) Applicant: Legend Biotech USA Inc., Somerset, NJ (US)

(72) Inventors: Fengyuan TANG, Nanjing (CN); Wang Zhang, Nanjing (CN); Qiuchuan ZHUANG, Nanjing (CN); Lei YANG, Nanjing (CN); Xiao YANG, Nanjing (CN); Qianqian LIU, Nanjing (CN); Rui GAO, Nanjing (CN)

(21) Appl. No.: 19/483,890

(22) PCT Filed: Jun. 7, 2024
(86) PCT No.: PCT/CN2024/098025

§ 371 Date: Nov. 12, 2025

(30) Foreign Application Priority Data
Jun. 9, 2023 (WO) PCT/CN2023/099457

(51) Int. Cl. C12N 5/0783; A61K 40/11; A61K 40/31; A61K 40/42; A61P 35/00; C07K 14/47; C07K 16/28

(52) U.S. Cl. CPC C12N 5/0636; A61K 40/11; A61K 40/31; A61K 40/4202; A61P 35/00; C07K 14/4702; C07K 16/28; C07K 2317/53; C07K 2317/569; C07K 2319/02; C07K 2319/03; C12N 2510/00

(57) ABSTRACT

Provided are engineered cells with enhanced transcriptional activities of a transcriptional enhanced associate domain (TEAD) protein, and methods of making and use thereof. The engineered cells overexpress a TEAD coactivator (e.g., a YAP peptide or a WWTR1 peptide).

### CROSS REFERENCE

This application claims benefit of priority of International Patent Application No. PCT/CN2023/099457 filed on Jun. 9, 2023, the content of which is incorporated herein by reference in its entirety.

### SEQUENCE LISTING

This application incorporates by reference a Sequence Listing submitted with this application as XML file format, entitled “IEC240103PCT SEQUENCE LISTING.xml,” created on Jun. 6, 2024 having a size of 33,578 bytes.

### TECHNICAL FIELD

This disclosure relates to engineered cells with enhanced transcriptional activities of a transcriptional enhanced associate domain (TEAD) protein, and methods of making and use thereof.

### BACKGROUND

Adoptive immunotherapy involves administration of immune cells to patients having cancer, tumor, or infections, whereby the administered immune cells provide a therapeutic benefit to the patients. Generally speaking, immune cells suitable for immunotherapy include, but are not limited to, B cells, dendritic cells (DC), T cells, Natural Killer (NK) cells, NKT (Natural Killer T) cells, and hematopoietic stem or progenitor cells. A central goal of these cell-based immunotherapies is to mediate complete and durable therapeutic effects in patients is.

The proliferative and survival capacities, migration to the tumor site(s), and the ability to sustain effector functions within the tumor are crucial determinants for triggering the eradication of malignant cells. However, even though these desirable attributes were recognized, the pathways or players driving these attributes are still unclear, which limits one's ability to intervene and obtain cells having desired therapeutic effects. Thus, there is a need for engineered cells (e.g., T cells, NK cells or NKT cells) with improved survival capacities, improved migration to the tumor site(s), and improved ability to sustain effector functions within the tumor.

### SUMMARY

This disclosure relates to engineered cells with enhanced TEAD protein transcriptional activities, and methods of making and use thereof.

In one aspect, this disclosure relates to engineered cells overexpressing a TEAD coactivator (e.g., a YAP peptide or a WWTR1 peptide), and methods of use thereof.

In some embodiments, the TEAD coactivator is Yes-Associated Protein (YAP).

In some embodiments, the TEAD coactivator is WW domain containing transcription regulator 1 (WWTR1), also known as Transcriptional co-activator with PDZ-binding motif (TAZ).

In one aspect, the disclosure is related to an engineered cell having an enhanced TEAD (transcriptional enhanced associate domain) protein transcriptional activity.

In some embodiments, the transcriptional activities of a TEAD coactivator are increased.

In some embodiments, the TEAD coactivator is selected from the group consisting of Yes-associated protein (YAP), WW domain-containing transcription regulator protein 1 (WWTR1), Vestigial like family member 4 (VgLL) and p160 coactivators (SRC-1, GRIP1/TIF2, and pCIP/RAC3/ACTR/AIB1/TRAM1).

In some embodiments, TEAD coactivator is a YAP peptide or a WWTR1 peptide.

In some embodiments, the engineered cell overexpresses a YAP peptide or a WWTR1 peptide.

In some embodiments, activities of an upstream regulator of the Hippo pathway are modulated, wherein the upstream regulator is selected from the group consisting of mammalian STE20-like protein kinases 1 and 2, and large tumor suppressor homologs 1 and 2 (LATS1/2), human Mps one binder 1A or 1B (MOB1A/B), human Savaldor (Sav1).

In one aspect, the disclosure is related to an engineered cell overexpressing a YAP peptide or a WWTR1 peptide.

In some embodiments, the engineered cell further expresses an engineered receptor.

In one aspect, the disclosure is related to an engineered cell comprising: an engineered receptor; and an exogenous YAP peptide or an exogenous WWTR1 peptide.

In some embodiments, the YAP peptide is a wildtype YAP peptide or a mutant YAP peptide; and the WWTR1 peptide is a wildtype WWTR1 peptide or a mutant WWTR1 peptide.

In some embodiments, the YAP peptide comprises an amino acid sequence of SEQ ID NO: 1 or SEQ ID NO: 2, or that is at least 95%, 99%, or 100% identical to the amino acid sequence set forth in SEQ ID NO: 1 or SEQ ID NO: 2.

In some embodiments, the WWTR1 peptide comprises an amino acid sequence of SEQ ID NO: 3 or SEQ ID NO: 4, or that is at least 95%, 99%, or 100% identical to the amino acid sequence set forth in SEQ ID NO: 3 or SEQ ID NO: 4.

In some embodiments, the engineered receptor is selected from the group consisting of an engineered T cell receptor (TCR), a chimeric antigen receptor (CAR), a T cell antigen coupler (TAC) or a portion thereof.

In some embodiments, the engineered receptor is a CAR.

In some embodiments, the CAR comprises an extracellular antigen binding domain that specifically binds to an antigen.

In some embodiments, the antigen is a tumor antigen selected from the group consisting of BCMA, CLL1, CD4, GPC3, GPRC5D, GU2CYC, CD19, MUC16, MUC1, CAIX, CEA, CD8, CD7, CD10, CD20, CD22, CD30, CD33, CD34, CD38, CD41, CD44, CD49f, CD56, CD74, CD133, CD138, EGP-2, EGP-40, EpCAM, ERBB2, ERBB3, ERBB4, FBP, fetal acetylcholine receptor, folate receptor-α, GD2, GD3, HER-2, hTERT, IL-13R-α2, κ-light chain, KDR, LeY, L1 cell adhesion molecule, MAGE-A1, mesothelin, MAGEA3, p53, MART1, GP100, proteinase-3 (PR3), tyrosinase, survivin, hTERT, EphA2, NY-ESO-1, h5T4, PSCA, PSMA, ROR1, TAG-72, VEGF-R2, WT-1, CD123, CD44V6, NKCS1, IGF1R, EGFR, EGFR-VIII, Claudin 18.2, Claudin 6, NKG2D, Delta-like 3 (DLL3), CD70, CS-1, c-Met, Glycolipid F77, PD-L1, and PD-L2.

In some embodiments, the antigen is DLL3.

In some embodiments, the CAR comprises an amino acid sequence of SEQ ID NO: 12, or an amino acid sequence that is at least 95%, 99%, or 100% identical to the amino acid sequence of SEQ ID NO: 12.

In some embodiments, the engineered cell comprises a polypeptide having an amino acid sequence set forth in any one of SEQ ID NOs: 13-16, or an amino acid sequence that is at least 95%, 99%, or 100% identical to the amino acid sequence set forth in any one of SEQ ID NOs: 13-16.

In some embodiments, the overexpression of the YAP peptide or the WWTR1 peptide is controlled by a non-native regulatory element.

In one aspect, the disclosure is related to a nucleic acid, comprising one or more nucleic acid sequences encoding: an engineered receptor; and/or a YAP peptide or WWTR1 peptide, wherein the YAP peptide or WWTR1 peptide is overexpressed.

In some embodiments, the nucleic acid sequence comprising one, two, three, four, five or more copies of the coding sequence of a YAP peptide or a WWTR1 peptide; or wherein the overexpression of the YAP peptide or the WWTR1 peptide is controlled by a non-native regulatory element.

In some embodiments, the YAP peptide comprises an amino acid sequence of SEQ ID NO: 1 or SEQ ID NO: 2, or an amino acid sequence that is at least 95%, 99%, or 100% identical to the amino acid sequence set forth in SEQ ID NO: 1 or SEQ ID NO: 2.

In some embodiments, the WWTR1 peptide comprises an amino acid sequence of SEQ ID NO: 3 or SEQ ID NO: 4, or an amino acid sequence that is at least 95%, 99%, or 100% identical to the amino acid sequence set forth in SEQ ID NO: 3 or SEQ ID NO: 4.

In some embodiments, the nucleic acid sequence encoding the engineered receptor and the nucleic acid sequence encoding the YAP peptide or the WWTR1 peptide are separated by a third nucleic acid sequence encoding a cleavable linker.

In some embodiments, the cleavable linker comprises an amino acid sequence set forth in SEQ ID NO: 17 or SEQ ID NO: 18, or a functional variant having at least about 90% sequence identity to the amino acid sequence of SEQ ID NO: 17 or SEQ ID NO: 18.

In some embodiments, the engineered receptor is selected from the group consisting of an engineered T cell receptor (TCR), a chimeric antigen receptor (CAR), a T cell antigen coupler (TAC) or a portion thereof.

In some embodiments, the engineered receptor is a CAR.

In some embodiments, the CAR comprises an amino acid sequence of SEQ ID NO: 12, or an amino acid sequence that is at least 95%, 99%, or 100% identical to the amino acid sequence of SEQ ID NO: 12.

In some embodiments, the nucleic acid sequence encodes an amino acid sequence set forth in any one of SEQ ID NOs: 13-16, or an amino acid sequence that is at least 95%, 99%, or 100% identical to an amino acid sequence set forth in any one of SEQ ID NOs: 13-16.

In one aspect, the disclosure is related to a vector comprising the nucleic acid described herein.

In one aspect, the disclosure is related to an engineered cell comprising the nucleic acid described herein, or the vector described herein.

In some embodiments, the engineered cell is an immune cell.

In some embodiments, the immune cell is selected from a group consisting of T cell, αβT cell, γδT cell, NK cell, NKT, peripheral blood mononuclear cell (PBMC), hematopoietic stem cell, pluripotent stem cell, an embryonic stem cell, and a combination thereof.

In some embodiments, the immune cell is a T cell.

In one aspect, the disclosure is related to a pharmaceutical composition comprising the engineered cell described herein, and a pharmaceutically acceptable carrier.

In one aspect, the disclosure is related to a method for producing the engineered immune cell described herein, the method comprising introducing the vector described herein into a cell.

In one aspect, the disclosure is related to a method of treating a disease or disorder in a subject, the method comprising administering to the subject, an effective amount of the engineered cell described herein, or the pharmaceutical composition described herein.

In some embodiments, the disease or disorder is cancer, autoimmune disease, or infection.

In some embodiments, the disease or disorder is solid tumor.

In some embodiments, the disease or disorder is selected from a group consisting of small cell lung cancer (SCLC), large cell neuroendocrine cancer (LCNC), neuroendocrine prostate cancer (NEPC), pancreatic neuroendocrine tumor (PNET) and gastrointestinal neuroendocrine cancers.

In some embodiments, the engineered cells overexpressing the YAP peptide and/or the WWTR1 peptide exhibit improved in vivo persistence relative to in vivo persistence of cells that do not overexpress the YAP peptide or the WWTR1 peptide.

Other features and advantages of the disclosure will be apparent from the following detailed description and figures, and from the claims.

### DESCRIPTION OF DRAWINGS

FIG. 1 shows the design scheme of vectors encoding DLL3 bi-specific chimeric antigen receptor (DLL3 CAR) and YAP or WWTR1 overexpressing DLL3 CARs. YAP and/or WWTR1 are derived from wildtype (Y wt and T wt) or mutant proteins (Y mut and T mut). CD8α SP, CD8α Hinge, CD8α TM, 4-1BB and CD3ζ stands for CD8α signal peptide, CD8α hinge domain, CD8α transmembrane domain, 4-1BB (CD137) co-stimulatory signaling domain and CD3ζ primary intracellular signaling domain, respectively.

FIGS. 2A-2D show the expression of membrane DLL3 CAR and intracellular wildtype or mutant YAP expression in the lentivirus infected primary T cells.

FIGS. 3A-3D show the expression of membrane DLL3 CAR and intracellular wildtype or mutant WWTR1 in the lentivirus infected primary T cells.

FIGS. 4A-4B show the cytotoxicity and cytokine release analysis of T cells infected with different CAR vectors against SHP-77 and NCI-H82 tumor cells at different E:T ratios. DLL3 CAR T cells, YAP or WWTR1 overexpressing DLL3 CAR T cells were co-cultured with SHP-77 (FIG. 4A) or NCI-H82 (FIG. 4B) at an E:T ratio of 1:1 and/or 0.3:1, respectively.

FIGS. 5A-5D show IFN-γ release after 24 hours of co-culture with target tumor cells SHP-77 (FIG. 5A) or NCI-H82 (FIG. 5C) at different E:T ratios, respectively. IFN-γ release by CAR T cells alone was monitored to interpret the CAR tonic activity (FIG. 5B and FIG. 5D). A stands for blowing the lower limit of quantification (<LLOQ).

FIGS. 6A-6E show T cell persistence (FIG. 6A and FIG. 6D), CAR positive T cell expansion (FIG. 6B and FIG. 6E) and exhaustive differentiation in CAR positive T cells (FIG. 6C). CAR T cells and UnT cells were cultured with tumor cells at an E:T ratio of 1:4 in a repetitive tumor re-challenge assay, respectively.

FIG. 7 shows the invasion and persistence of CAR T cells in a 3D-tumor model. SHP-77 3D tumor model was established by co-culturing the tumor cells with Matrigel. Tumor stromal invasion and anti-tumor activities of DLL3 CAR T cells and YAP or WWTR1 overexpressing DLL3 CAR T cells were analyzed.

FIGS. 8A-8D show in vivo efficacy and pharmacokinetics of conventional DLL3 CAR T cells and wildtype or mutant WWTR1 armored DLL3 CAR T cells against an xenograft model derived from SCLC tumor cell line NCI-H82. Tumor growth (FIG. 8A), animal survival curve (FIG. 8B) and body weight was recorded for monitoring safety properties of CAR T products (FIG. 8C). CAR T percentage in peripheral blood was also determined (FIG. 8D).

FIG. 9 shows selected sequences listed in the present disclosure.

### DETAILED DESCRIPTION

The present disclosure is based on, in part, the finding that engineered immune cells that overexpress a TEAD coactivator peptide have enhanced persistence, and can be used in cell therapies. Transmitting the tumor-intrinsic pro-inflammatory traits can sustain the anti-tumor activity of adoptive transferred cells. Wildtype or constitutive active TEAD coactivator can be overexpressed in adoptive transferred cells, such as chimeric antigen receptor T (CAR-T) cells. In addition, the constitutive active YAP or WWTR1 is resistant to inactivation by LATS-mediated phosphorylation upon stresses conditions. The present disclosure further provides cell therapies with improved efficacy by leveraging the transcriptional outputs mediated by a TEAD coactivator (e.g., a YAP peptide or a WWTR1 peptide).

In one aspect, the disclosure provides an engineered cell having an enhanced TEAD protein transcriptional activity. The transcriptional activities of a TEAD coactivator may be increased. The TEAD coactivator may be selected from the group consisting of Yes-associated protein (YAP), WW domain-containing transcription regulator protein 1 (WWTR1), Vestigial like family member 4 (VgLL) and p160 coactivators (SRC-1, GRIP1/TIF2, and pCIP/RAC3/ACTR/AIB1/TRAM1). The TEAD coactivator may be YAP or WWTR1. The engineered cell may overexpress a YAP peptide or a WWTR1 peptide. The activities of an upstream regulator of the Hippo pathway may also be modulated. The upstream regulator may be selected from the group consisting of mammalian STE20-like protein kinases 1 and 2, and large tumor suppressor homologs 1 and 2 (LATS1/2), human Mps one binder 1A or 1B (MOB1A/B), human Savaldor (Sav1).

In one aspect, the disclosure provides a method to enhance the transcriptional activities of a TEAD protein. This can be achieved by enhancing the transcriptional activities of a TEAD coactivator (e.g., by overexpressing a TEAD coactivator). Some exemplary TEAD coactivators include: YAP, WWTR1 (TAZ), Vestigial like family member 4 (VgLL) and p160 coactivators (SRC-1, GRIP1/TIF2, and pCIP/RAC3/ACTR/AIB1/TRAM1). The method may include overexpressing a YAP peptide or a WWTR1 peptide. The method may include reducing the activities of an upstream regulator of the Hippo pathway. The upstream regulator may be selected from the group consisting of mammalian STE20-like protein kinases 1 and 2, and large tumor suppressor homologs 1 and 2 (LATS1/2), human Mps one binder 1A or 1B (MOB1A/B), human Savaldor (Sav1).

In one aspect, the disclosure provides engineered cells overexpressing a YAP peptide or a WWTR1 peptide. The overexpression of the YAP peptide or the WWTR1 peptide can be achieved by introducing into the cells a vector encoding the YAP peptide or the WWTR1 peptide. The overexpression of the YAP peptide or the WWTR1 peptide can also be achieved by modulating the activities of an upstream regulator of the Hippo pathway (e.g., LATS1/2). The YAP peptide or the WWTR1 peptide may be endogenous or exogenous. The YAP peptide or the WWTR1 peptide may be expressed from an exogenous vector.

In one aspect, the disclosure provides nucleic acids that encode a YAP peptide and/or a WWTR1 peptide and/or an engineered receptor.

In one aspect, the disclosure provides compositions and methods that result in increased longevity and persistence of effector immune cells (e.g., CAR-T or CAR-NK) in the body of a patient. Specifically, the disclosure provides effector immune cells (e.g., CAR-T cells) and methods that provide persistence (e.g., protection against immune clearance) and curative effect (e.g., cytotoxicity).

As used herein, the term “overexpress” generally refers to any amount greater than or equal to an expression level exhibited by a reference standard. The terms “overexpress,” “overexpressing,” “overexpressed” and “overexpression” in the present disclosure refer an expression of a gene product or a polypeptide at a level greater than the expression of the same gene product or polypeptide prior to a genetic alteration of the host cell or in a comparable host which has not been genetically altered at defined conditions. If a host cell does not comprise a given gene product, it is possible to introduce the gene product into the host cell for expression; in this case, any detectable expression is encompassed by the term “overexpression.”

As used herein, the term “YAP peptide” refers to a polypeptide derived from a wild-type YAP, the isoform of YAP, or a functional variant thereof. The YAP peptide can be a wild-type YAP (e.g., human YAP) peptide. The YAP peptide can be a mutant YAP peptide having one or more mutations (e.g., insertions, deletions, and/or substitutions).

As used herein, the term “WWTR1 peptide” refers to a polypeptide derived from a wild-type WWTR1, the isoform of WWTR1, or a functional variant thereof. The WWTR1 peptide can be a wild-type WWTR1 (e.g., human WWTR1) peptide. The WWTR1 peptide can be a mutant WWTR1 having one or more mutations (e.g., insertions, deletions, and/or substitutions).

As used herein, a “vector” is any construct capable of delivering one or more nucleic acids of interest to a host cell when the vector is introduced to the host cell. An “expression vector” is capable of delivering and expressing the one or more nucleic acids of interest as an encoded polypeptide in a host cell into which the expression vector has been introduced. Thus, in an expression vector, the nucleic acid of interest is positioned for expression in the vector by being operably linked with regulatory elements such as a promoter, enhancer, and/or a poly-A tail, either within the vector or in the genome of the host cell at or near or flanking the integration site of the nucleic acid of interest such that the nucleic acid of interest will be translated in the host cell introduced with the expression vector.

As used herein, the term “chimeric antigen receptor” or “CAR” as used herein refers to genetically engineered receptors, which can be used to graft one or more antigen specificity onto immune effector cells, such as T cells. Some CARs are also known as “artificial T-cell receptors,” “chimeric T cell receptors,” or “chimeric immune receptors.” A CAR may comprise an extracellular ligand binding domain or an extracellular antigen binding domain specific for one or more ligands or antigens (such as tumor antigens), a transmembrane domain, and an intracellular signaling domain. “CAR-T cell” refers to a T cell that expresses a CAR.

As used herein, the term “T-cell receptor” or “TCR” as used herein refers to an endogenous or modified T-cell receptor comprising an extracellular antigen binding domain that binds to a specific antigenic peptide bound in an MHC molecule. The TCR may comprise a TCRα polypeptide chain and a TCRβ polypeptide chain. The TCR may comprise a TCRγ polypeptide chain and a TCRδ polypeptide chain. The TCR may specifically bind to a tumor antigen. “TCR-T” refers to a T cell that expresses a recombinant TCR. Expression of a heterologous antigen receptor, such as a heterologous TCR or CAR, can alter the immunogenic specificity of the T cells so that they recognize or display improved recognition for one or more tumor antigens that are present on the surface of the cancer cells of an individual with cancer.

As used herein, the term “T cell antigen coupler (TAC)” refers to an engineered receptor that redirects the endogenous T cell receptor (TCR) against a tumor target via an extracellular antigen binding domain. It may comprise (1) an extracellular antigen binding domain, (2) a TCR-recruitment domain.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, endocrine systems and neuroendocrine tumor (NET); as well as adenocarcinomas which include malignancies such as most colon cancers, renal-cell carcinoma, prostate cancer and/or testicular tumors, small cell lung cancer (SCLC), large cell neuroendocrine cancer (LCNC), neuroendocrine prostate cancer (NEPC), pancreatic neuroendocrine tumor (PNET), gastrointestinal neuroendocrine cancers, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### YAP/WWTR1 Peptide

Yes-Associated Protein (YAP) and WW domain containing transcription regulator 1 (WWTR1; also known as TAZ) are the transcriptional co-regulators and downstream effectors of the Hippo signaling pathway. The Hippo pathway play an essential role in cell and tissue physiology in normal tissue. It is also crucial in cancer origin and progression. YAP and WWTR1 promote tumor development, progression, and metastasis. At cellular level, YAP and WWTR1 regulate cell cycles, migration, invasion, anchorage-independent growth, epithelial-mesenchymal transition (EMT), and stemness. Because all these functions of YAP and WWTR1 are crucial for various steps of metastasis, YAP and WWTR1 serve as a central regulator of metastasis. Besides, it has gradually been recognized that YAP and WWTR1 regulate cellular metabolic reprograming to adapt the various tumor microenvironment.

YAP and WWTR1 are co-transcriptional coactivators with alternating locations, the cytoplasm and the nucleus. Both have a similar structural composition, sharing over half of their amino acid sequences and characteristics. YAP and WWTR1 are multidomain proteins that interact with several other proteins, and their WW domains allow them to bind, for example, to proteins containing a PPxY motif. They also interact with TEAD through regions that have been mapped within their N terminal, and they promote oncogenic transformation in some cancers.

The critical steps of the Hippo pathway are represented by YAP and WWTR1. The YAP/WWTR1 is phosphorylated in the cytoplasm and unphosphorylated in the nucleus. Upon dephosphorylation, active YAP/TAZ translocate to nucleus and interact majorly with TEA domain family member (TEAD) transcription factors (TEAD1-4). In nucleus, the YAP/TAZ-TEAD protein complex transcribes genes that control cell proliferation, apoptosis and cell fate. Abnormal nuclear expression of YAP and/or WWTR1 proteins is associated with poor prognosis of different types of cancer. In addition, YAP and/or WWTR1 can associate with other transcription factors leading to cell remodeling, proliferation, and invasion.

TEAD proteins (TEADs) are the downstream effectors of the Hippo signaling pathway that regulate cell proliferation and stem cell functions. All members of the TEAD family share a highly conserved DNA binding domain called the TEA domain. TEADs are unable to activate transcription and require the help of coactivators such as YAP, WWTR1, VgLL, and p160 proteins. The expression of TEAD family is up-regulated in many cancer types including gastric, colorectal, breast, and prostate cancers, which is correlated with poor survival in patients. Pharmacological modulators of TEADs could therefore find application in cancer treatment and regenerative medicine.

The YAP gene is located in the 11q22 chromosome in humans. The central region of the protein comprises: (1) one or more WW domains, (2) a transactivation domain on its C-terminal region, (3) a double spiral domain, and (4) a PDZ-binding motif. This gene has eight isoforms classified into two large groups: YAP1 and YAP2. The product differs in the number of WW domains.

The WWTR1 gene is located in the 3q23-q24 chromosome, and it is abundantly expressed in different tissues. There are three variants in humans formed by one WW domain.

Unlike YAP, WWTR1 does not have an Src homology 3 (SH3) binding motif nor a double spiral domain.

A detailed review of YAP and WWTR1 and their functions can be found in Piccolo, Stefano, Sirio Dupont, and Michelangelo Cordenonsi. “The biology of YAP/TAZ: hippo signaling and beyond.” Physiological reviews (2014); Yamaguchi, Hirohito, and Ghina M. Taouk. “A potential role of YAP/TAZ in the interplay between metastasis and metabolic alterations.” Frontiers in oncology 10 (2020): 928; Wang, Lijuan, et al. “Overexpression of YAP and TAZ is an independent predictor of prognosis in colorectal cancer and related to the proliferation and metastasis of colon cancer cells.” PloS one 8.6 (2013): e65539; He, Lizhi, et al. “YAP and TAZ are transcriptional co-activators of AP-1 proteins and STAT3 during breast cellular transformation.” Elife 10 (2021): e67312; Ortega, Ángel, et al. “The YAP/TAZ signaling pathway in the tumor microenvironment and carcinogenesis: Current knowledge and therapeutic promises.” International Journal of Molecular Sciences 23.1 (2022): 430, each of which is incorporated by reference in its entirety.

In one aspect, the present disclosure provides engineered cells (e.g., CAR-T cells, CAR-NK cells, TCR-T cells) overexpressing a YAP peptide and/or a WWTR1 peptide. The YAP peptide and/or the WWTR1 peptide can enhance the persistence of the engineered cells. The YAP peptide and/or the WWTR1 peptide may be endogenous or exogenous. The YAP peptide and/or the WWTR1 peptide may be expressed from an exogenous vector.

The YAP peptide may be wildtype (wt) or mutant (mut). In some cases, the YAP peptide has an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1 or SEQ ID NO: 2. In some cases, the YAP peptide has an amino acid sequence that is identical to the amino acid sequence of SEQ ID NO: 1 or SEQ ID NO: 2. The YAP peptide can have 1, 2, 3, 4, 5, 6, 7, 8, 9, 10 or more than 10 mutations. Each mutation can be a deletion, an insertion, or a substitution.

The WWTR1 peptide may be wildtype (wt) or mutant (mut). In some cases, the WWTR1 peptide has an amino acid sequence that is at least 60%, 70%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 3 or SEQ ID NO: 4. In some cases, the WWTR1 peptide has an amino acid sequence that is identical to the amino acid sequence of SEQ ID NO: 3 or SEQ ID NO: 4. The WWTR1 peptide can have 1, 2, 3, 4, 5, 6, 7, 8, 9, 10 or more than 10 mutations. Each mutation can be a deletion, an insertion, or a substitution.

### Engineered Cells

In one aspect, the present disclosure provides engineered cells (e.g., engineered immune cells) with enhanced TEAD transcriptional activities. In one aspect, the present disclosure provides engineered cells that overexpress a TEAD coactivator (e.g., a YAP peptide or WWTR1 peptide).

In one aspect, the present disclosure provides engineered cells overexpressing the YAP peptide and/or the WWTR1 peptide described herein. The overexpression of the YAP peptide and/or the WWTR1 peptide may be controlled by a non-native regulatory element. The non-native regulatory element may be an exogenous promoter. The engineered cells overexpressing the YAP peptide and/or the WWTR1 peptide described herein can further express an engineered receptor (e.g., CAR). The engineered cells described herein can express more than one engineered receptor, such as any combination of CAR, TCR, or TAC receptors.

Examples of suitable non-native regulatory element include a cytomegalovirus (CMV) promoter, a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Inducible promoters are also contemplated as part of the disclosure. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The engineered cell overexpressing the YAP peptide and/or the WWTR1 peptide may have multiple copies of a nucleic acid sequence encoding the YAP peptide and/or the WWTR1 peptide. The engineered cell may comprise one, two, three, four, five or more copies of the coding sequence of the YAP peptide and/or the WWTR1 peptide. The engineered cell may express an over-active mutant YAP peptide and/or an over-active mutant WWTR1 peptide. The engineered cell may express a mutant upstream regulator of the Hippo pathway. The engineered cell may overexpress or under-express an upstream regulator of the Hippo pathway. The upstream regulator can be any one of mammalian STE20-like protein kinases 1 and 2, and large tumor suppressor homologs 1 and 2 (LATS1/2), human Mps one binder 1A or 1B (MOB1A/B), human Savaldor (Sav1).

The cell that is to be engineered can be obtained from e.g., humans or non-human animals. The cell that is to be engineered can be obtained from bacteria, fungi, humans, rats, mice, rabbits, monkeys, pig or any other species. For example, the cell can be obtained from humans, rats or mice. The cells may be mouse lymphocytes and engineered (e.g., transduced) to express the YAP peptide and/or the WWTR1 peptide described herein. The cell may be obtained from humans. The cell may be a blood cell. The cell may be a leukocyte (e.g., a T cell), lymphocyte or any other suitable blood cell type. The cell may be a peripheral blood cell. The cell may be a tumor-infiltrating lymphocyte (TIL). The cell may be a T cell, a B cell, an NKT cell or an NK cell. The cells may be human peripheral blood mononuclear cells (PBMCs). The human PBMCs may be CD3+ cells. The human PBMCs may be CD8+ cells or CD4+ cells.

The cell may be a T cell. The T cells may express one or more engineered receptors that recognize a specific antigen on the surface of a target cell. T cells can be obtained by various methods known in the art, e.g., in vitro culture of T cells (e.g., tumor infiltrating lymphocytes) isolated from subjects. Genetically engineered T cells can be obtained by transducing T cells (e.g., isolated from the peripheral blood of subjects), with a vector, such as the vector provided herein. The T cells may be CD4+ T cells, CD8+ T cells, or regulatory T cells. The T cells may be T helper type 1 T cells and/or T helper type 2 T cells. The T cell may be an αβ-T cell. The T cell expressing this receptor may be a γδ-T cell. The T cells may be central memory T cells. The T cells may be effector memory T cells. The T cells may be naïve T cells.

The preparation of the engineered immune cells may include one or more culture and/or preparation steps. The cells which are to be engineered can be isolated from a sample, such as a biological sample, e.g., one obtained from or derived from a subject. The subject from which the cell is isolated may have a disease or condition or need a cell therapy or to which cell therapy will be administered. The subject may be a human in need of a particular therapeutic intervention, such as an adoptive cell therapy for which the cells are being isolated, processed, and/or engineered.

The cells may be stem cells, such as hematopoietic stem cell, embryonic stem cell and pluripotent stem cell, including induced pluripotent stem cells (iPSCs). The cells can be primary cells, such as those isolated directly from a subject and/or isolated from a subject and frozen. The stem cells may be cultured with additional differentiation factors to obtain desired cell types (e.g., T cells).

Different cell types can be obtained via appropriate isolation methods. The isolation methods include the separation of different cell types based on the expression or presence in the cell of one or more specific molecules, such as surface markers, e.g., surface proteins, intracellular markers, or nucleic acid. Any known method for separation based on such markers can be used. The separation may be affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. Both fractions may be retained for further use. Negative selection can be particularly useful where no antibody is available that specifically identifies a cell type in a heterogeneous population, such that separation is best carried out based on markers expressed by cells other than the desired population.

Also provided are methods, nucleic acids, compositions, and kits, for expressing the YAP peptide and/or the WWTR1 peptide and/or the engineered receptor, and/or for producing the engineered immune cells. The genetic engineering generally involves introduction of a nucleic acid encoding the YAP peptide and/or the WWTR1 peptide into the cell, such as by retroviral transduction, transfection, or transformation. Gene transfer may be accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical application.

Recombinant nucleic acids may be transferred into cells using recombinant infectious virus particles, such as, e.g., vectors derived from simian virus 40 (SV40), adenoviruses, adeno-associated virus (AAV). Recombinant nucleic acids may be transferred into T cells using recombinant lentiviral vectors or retroviral vectors, such as gamma-retroviral vectors. The retroviral vector may have a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. The retroviruses may include those derived from any avian or mammalian cell source. The retroviruses typically are amphotropic, meaning that they are capable of infecting host cells of several species, including humans. The vector may be a lentivirus vector. In certain embodiments, the vector comprises any one of the nucleic acids encoding the YAP peptide and/or the WWTR1 peptide and/or the engineered receptor described herein. The nucleic acid can be cloned into the vector using any known molecular cloning methods in the art, including, for example, using restriction endonuclease sites and one or more selectable markers. Recombinant nucleic acids may be transferred into T cells via electroporation. Recombinant nucleic acids may be transferred into T cells via transposition. Other methods of introducing and expressing genetic material in immune cells include calcium phosphate transfection, protoplast fusion, cationic liposome-mediated transfection; tungsten particle-facilitated microparticle bombardment and strontium phosphate DNA co-precipitation. Many of these methods are descried e.g., in WO2019195486, which is incorporated herein by reference in its entirety. A T cells may be pre-activated, e.g., using anti-CD3/CD28 particles, for about 12 hours, about 24 hours, about 36 hours, about 48 hours, or about 60 hours prior to transduction. A transduced T cells may be harvested on day 5, day 6, day 7, day 8, day 9, day 10, day 11, or day 12 post transduction.

The engineered immune cells may be used to treat various disorder or disease as described herein (e.g., cancer).

Comparing to cells that do not overexpress the YAP peptide or the WWTR1 peptide described herein, the engineered cells overexpressing the YAP peptide and/or the WWTR1 peptide described herein may have a higher persistence and/or proliferation. Comparing to cells that do not overexpress the YAP peptide or the WWTR1 peptide described herein, the engineered cells overexpressing the YAP peptide and/or the WWTR1 peptide described herein may have a higher cytotoxicity against tumor cells. Comparing to cells that do not overexpress the YAP peptide or the WWTR1 peptide, the engineered cells overexpressing the YAP peptide and/or the WWTR1 peptide may have enhanced proliferation and/or persistence. Comparing to cells that do not overexpress the YAP peptide or the WWTR1 peptide, the engineered cells overexpressing the YAP peptide and/or the WWTR1 peptide may have enhanced cytotoxicity. Comparing to cells that do not overexpress the YAP peptide or the WWTR1 peptide, the engineered cells overexpressing the YAP peptide and/or the WWTR1 peptide may have reduced exhaustive differentiation.

The engineered cells described herein can be eukaryotic cells, e.g., mammalian cells. The engineered cells can be human cells. The engineered cells can be equine, bovine, murine, ovine, canine, or feline cells. The engineered cells can be modified immune cells. The engineered cells can be T cells. The engineered cells can be NK cells. The engineered cells can be αβ T cells. The engineered cells can be γδ T cells. The engineered cells can be V81 T cells.

The engineered cells can be autologous cells, syngeneic cells, allogeneic cells, or xenogeneic cells with respect to the individual receiving them. The engineered cells can be modified by changing the major histocompatibility complex (MHC) profile, by inactivating β2-microglobulin to prevent the formation of functional Class I MHC molecules, or by inactivating Class II MHC molecules. The engineered cells can be autologous cells obtained from the human subject receiving them. The engineered cells can be autologous T cells obtained from the human subject receiving them.

The engineered receptor can be a CAR. The CAR can comprise a polypeptide comprising from N-terminus to C-terminus: a CD8α signal peptide, an extracellular antigen binding domain, a CD8α hinge domain, a CD8α transmembrane domain, a 4-1BB co-stimulatory signaling domain, and a CD3ζ primary intracellular signaling domain. In some embodiments, the CAR is a DLL3 CAR. In some cases, the CAR comprises a polypeptide comprising an amino acid sequence having at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to in the amino acid sequence of SEQ ID NO: 12.

The YAP peptide can comprise an amino acid sequence having at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to in the amino acid sequence of SEQ ID NO: 1 or SEQ ID NO: 2.

The WWTR1 peptide can comprise an amino acid sequence having at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to in the amino acid sequence of SEQ ID NO: 3 or SEQ ID NO: 4.

The engineered cells may over express the YAP peptide and/or the WWTR1 peptide and express a CAR (CAR armored with the YAP peptide and/or the WWTR1 peptide). The engineered cells can be modified CAR-T cells (the YAP peptide and/or the WWTR1 peptide armored CAR-T cells).

The engineered cells may comprise a polypeptide comprising a CAR and a YAP peptide. The polypeptide may comprise from the N-terminus to the C-terminus: a CD8α signal peptide, an extracellular antigen binding domain, a CD8α hinge domain, a CD8α transmembrane domain, a 4-1BB co-stimulatory signaling domain, and a CD3ζ primary intracellular signaling domain, a 2A cleavable linker, and a YAP peptide. In some embodiments, the polypeptide comprising a DLL3 CAR and a wildtype YAP peptide is referred to as “DLL3 CAR Y wt”. The amino acid sequence can be at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 13. In some embodiments, the polypeptide comprising a DLL3 CAR and a mutant YAP peptide is referred to as “DLL3 CAR Y mut”. The amino acid sequence can be at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 14.

The engineered cells may comprise a polypeptide comprising a CAR and a WWTR1 peptide. The polypeptide may comprise from the N-terminus to the C-terminus: a CD8α signal peptide, an extracellular antigen binding domain, a CD8α hinge domain, a CD8α transmembrane domain, a 4-1BB co-stimulatory signaling domain, and a CD3ζ primary intracellular signaling domain, a 2A cleavable linker, and a WWTR1 peptide. In some embodiments, the polypeptide comprising a DLL3 CAR and a wildtype WWTR1 peptide is referred to as “DLL3 CAR T wt”. The amino acid sequence can be at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 15. In some embodiments, the polypeptide comprising a DLL3 CAR and a mutant WWTR1 peptide is referred to as “DLL3 CAR T mut”. The amino acid sequence can be at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 16.

The expression of CAR and the YAP peptide and/or the WWTR1 peptide by the engineered cells can be determined by flow cytometry (FACS) (e.g., following the methods described in Example 2). The engineered cells can have a CAR positive rate of more than 5%, more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, or more than 90%. The engineered cells can have a CAR positive rate of less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, or less than 90%. In some cases, the engineered cells have a CAR positive rate of 20%-50%, 20%-40%, or 25%-40%.

The YAP peptide positive rate and the WWTR1 peptide positive rate of the engineered cells can be determined by flow cytometry (FACS) (e.g., following the methods described in Example 2). The engineered cells can have a YAP peptide positive rate or a WWTR1 peptide positive rate of more than 5%, more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, or more than 90%. The engineered cells can have a YAP peptide positive rate or a WWTR1 positive rate of less than 5%, less than 10%, less than 15%, less than 20%, less than 25%, less than 30%, less than 35%, less than 40%, less than 45%, less than 50%, less than 55%, less than 60%, less than 65%, less than 70%, less than 75%, less than 80%, or less than 90%. In some cases, the engineered cells have a YAP peptide positive rate or a WWTR1 peptide positive rate of 10%-50%, 10%-40%, or 15%-40%.

The populations of engineered cells, compositions containing such cells and/or enriched for such cells, may make up at least 15%, 20%, 25%, 30%, 35%, 40%, 50%, 60%, 70%, 80%, 90%, 91%, 92%, 93%, 94%, 95%, 96%, 97%, 98%, 99%, or more percent of the total cells in the composition or cells of a certain type such as T cells, CD8+ or CD4+ cells.

The viability of the transduced T cells may be at least 85%, at least 86%, at least 87%, at least 88%, at least 89%, at least 90%, at least 91%, at least 92%, at least 93%, at least 94%, or at least 95% on day 0, day 1, day 2, day 3, day 4, or day 5 post transduction. The viability of the transduced T cells may be at least or about 80%, at least or about 90%, at least or about 100%, at least or about 110%, at least or about 120% as compared to the viability of un-transduced T cells, on day 0, day 1, day 2, day 3, day 4, or day 5 (e.g., on day 5) post transduction.

The T cell expansion fold may be at least 1 fold, 2 folds, 3 folds, 4 folds, 5 folds, 10 folds, 15 folds, 20 folds, 25 folds, 30 folds, 35 folds, 40 folds, 45 folds, or 50 folds, on day 0, day 1, day 2, day 3, day 4, or day 5 post transduction. The T cell expansion fold of the transduced T cells may be at least or about 50%, at least or about 60%, at least or about 70%, at least or about 80%, at least or about 90% higher as compared to that of un-transduced T cells, on day 0, day 1, day 2, day 3, day 4, or day 5 (e.g., on day 5) post transduction.

The engineered cells may kill tumor cells. The cytotoxicity of the engineered cells against tumor cells (e.g., SHP-77 cells or NCI-H82 cells) can be determined by an in vitro cytotoxicity assay (e.g., following the methods described in Example 3). The engineered cells (e.g., CAR-T cells) may be co-cultured with target cells for at least or about 1 hour, 2 hours, 3 hours, 4 hours, 5 hours, 6 hours, 12 hours, 16 hours, 18 hours, 1 day, 2 days, 3 days, or longer, such that the engineered cells (e.g., CAR-T cells) can be activated. The effector cell: target cell (E: T) ratio can be 0.3:1, 0.5:1, 1:1, 2:1, 2.5:1, 5:1, or 10:1. The E: T ratio can be 0.3:1 or 1:1. The engineered cells may have a cytotoxicity of more than 5%, more than 10%, more than 15%, more than 20%, more than 30%, more than 40%, more than 50%, more than 60%, more than 70%, more than 80%, or more than 90%. The engineered cells may have a cytotoxicity of less than 5%, less than 10%, less than 15%, less than 20%, less than 30%, less than 40%, less than 50%, less than 60%, less than 70%, less than 80%, or less than 90%. In some cases, the engineered cells have a cytotoxicity of 10-100%, 10%-50%, 20-100%, 20-60%, 20-40%, 30-70%, 40-80%, 50-90%, 70-100%, 80-100%, or 90-100%. The cytotoxicity may be specific for the target cells that express a target antigen, for example DLL3. The engineered cells may induce little or background cytotoxicity against target cells which do not express the target antigen (e.g., cells that do not express DLL3).

Engineered cells overexpressing the YAP peptide and/or the WWTR1 peptide may have in vitro cytotoxicity, as measured by the method described in the Examples, which is higher than immune cells that do not overexpress the YAP peptide or the WWTR1 peptide, when the engineered cells are exposed to target cells comprising a target antigen, for example at least 10%, at least 20%, at least 30% higher, or at least 60% higher. Beneficially higher cytotoxicity may lead to more target tumor cells being killed.

Engineered cells expressing a mutant YAP peptide or a mutant WWTR1 peptide (e.g., SEQ ID NO: 2 or SEQ ID NO: 4), may have lower cytotoxicity as measured according to the method of the Examples compared to engineered cells expressing a wildtype YAP peptide or wildtype WWTR1 peptide (e.g., SEQ ID NO: 1 or SEQ ID NO: 3), when the engineered cells are exposed to target cells comprising a target antigen, for example at least 15% lower. It may be beneficial to lower the cytotoxicity of the engineered cells as this may reduce the risk of a cytokine storm by controlling immune cell activation. Engineered cells expressing a mutant YAP peptide or a mutant WWTR1 peptide (e.g., SEQ ID NO: 2 or SEQ ID NO: 4), may have higher cytotoxicity as measured according to the method of the Examples compared to engineered cells expressing a wildtype YAP peptide or wildtype WWTR1 peptide (e.g., SEQ ID NO: 1 or SEQ ID NO: 3), when the engineered cells are exposed to target cells comprising a target antigen, for example at least 15% higher.

The long-term cytotoxicity of the engineered cells (e.g., CAR-T cells) can be determined, e.g., by re-challenging the engineered cells. Exemplary re-challenging procedures of CAR-T cells can be found, e.g., in Wang, Dongrui, et al., Journal of Visualized Experiments: JoVE 144 (2019); Wang D, et al., JCI Insight 2018, 3 (10); Lange et al., Cancer Discov. 2021 Feb. 9, candisc.0896.2020; each of which is incorporated herein by reference in its entirety.

The engineered cells may secrete cytokines (e.g., IFNγ), with or without exposure to antigens to which the antigen binding domains recognize. Cytokine release can be measured in an in vitro cytokine release assay (e.g., following the methods described in Example 4). Concentration of the cytokines (e.g., IFN-γ) released by the engineered cells (e.g., CAR-T cells) described herein can be determined by homogeneous time resolved fluorescence (HTRF) assays. The engineered cells may be co-cultured with tumor cells (e.g., SHP-77 cells or NCI-H82 cells). The effector cell: target cell (E: T) ratio may be 0.5:1, 1:1, 2:1, 2.5:1, 5:1, or 10:1. The E: T ratio may be 0.3:1 or 1:1. The engineered cells may secrete an amount of IFNγ that is more than 50 pg/ml, more than 100 pg/ml, more than 200 pg/ml, more than 300 pg/ml, more than 400 pg/ml, more than 500 pg/ml, more than 1000 pg/ml, more than 1500 pg/ml, more than 2000 pg/ml, more than 2500 pg/ml, or more than 3000 pg/ml. The engineered cells may secrete an amount of IFNγ that is less than 50 pg/ml, less than 100 pg/ml, less than 200 pg/ml, less than 300 pg/ml, less than 400 pg/ml, less than 500 pg/ml, less than 1000 pg/ml, less than 1500 pg/ml, less than 2000 pg/ml, less than 2500 pg/ml, or less than 3000 pg/ml. In some cases, the engineered cells may secrete an amount of IFNγ that is 500-5000 pg/ml, 1000-4000 pg/ml, 1000-3000 pg/ml, 1500-3000 pg/ml, 500-3000 pg/ml, 500-2500 pg/ml, 50-500 pg/ml, 50-400 pg/ml, 100-1000 pg/ml, 100-800 pg/ml, 100-600 pg/ml, 100-400 pg/ml, 200-400 pg/ml, 200-300 pg/ml, 200-1000 pg/ml, or 200-800 pg/ml. In some cases, comparing to cells that do not overexpress the YAP peptide or the WWTR1 peptide, the cytokine secretion of the engineered cells overexpressing the YAP peptide and/or the WWTR1 peptide does not change significantly. The engineered immune cells overexpressing the YAP peptide and/or the WWTR1 peptide may secrete less IFNγ comparing to cells that do not overexpress the YAP peptide or the WWTR1 peptide. The engineered cells may secrete more IFNγ comparing to un-transfected control cells. The engineered cells may have an increased cytokine (e.g., IFN-γ) expression or secretion compared to wildtype, i.e. non-engineered cells. The cytokine release may increase by at least or about 1 fold, 2 folds, 3 folds, 4 folds, 5 folds, 10 folds, 20 folds, 30 folds, 40 folds, 50 folds, 60 folds, 70 folds, 80 folds, 90 folds, 100 folds, 500 folds, 1000 folds, 2000 folds, 3000 folds, 4000 folds, 5000 folds, or 10000 folds.

Engineered cells overexpressing the YAP peptide and/or the WWTR1 peptide may have in vitro IFNγ production, as measured by the method described in the Examples, which is higher than cells that do not overexpress the YAP peptide or the WWTR1 peptide, when the engineered cells are exposed to target cells comprising a target antigen. Engineered cells overexpressing the YAP peptide and/or the WWTR1 peptide may have in vitro IFNγ production of at least about 2000 pg/ml when exposed to target cells at an E:T ratio of 1:1 according to the method of the Examples. Engineered cells overexpressing the YAP peptide and/or the WWTR1 peptide may have in vitro IFNγ production of less than about 1000 pg/ml when exposed to target cells at an E:T ratio of 0.3:1 according to the method of the Examples.

Engineered cells expressing a mutant YAP peptide or a mutant WWTR1 peptide (e.g., SEQ ID NO: 2 or SEQ ID NO: 4), may have lower in vitro IFNγ production as measured according to the method of the Examples compared to engineered cells expressing a wildtype YAP peptide or a wildtype WWTR1 (e.g., SEQ ID NO: 1 or SEQ ID NO: 3), when the engineered cells are exposed to target cells comprising a target antigen, for example at least 15% lower. It may be beneficial to lower the in vitro IFNγ production of the engineered cells as this may reduce the risk of a cytokine storm. Engineered cells expressing a mutant YAP peptide or a mutant WWTR1 peptide (e.g., SEQ ID NO: 2 or SEQ ID NO: 4), may have higher in vitro IFNγ production as measured according to the method of the Examples compared to engineered cells expressing a wildtype YAP or a wildtype WWTR1 peptide (e.g., SEQ ID NO: 1 or SEQ ID NO: 3), when the engineered cells are exposed to target cells comprising a target antigen, for example at least 15% higher.

Comparing to cells that do not overexpress the YAP peptide or the WWTR1 peptide described herein, the engineered cell overexpressing the YAP peptide and/or the WWTR1 peptide described herein can have an improved persistence and/or expansion. Cell persistence, exhaustion, and expansion can be measured by repeatedly stimulating the engineered cells with tumor cells (e.g., SHP-77 cells) for several rounds in an in vitro re-challenge assay (e.g., following the methods described in Example 5). The engineered cells can be first challenged (co-cultured) with tumor cells (e.g., SHP-77 cells) for a first round, and then re-challenged with fresh tumor cells (e.g., SHP-77 cells) in a subsequent round. The effector cell: target cell (E: T) ratio can be 1:4, 0.5:1, 1:1, 2:1, 2.5:1, 5:1, or 10:1. In some cases, the E:T ratio is 1:4. The engineered cells can be exposed to 1 round, 2 rounds, 3 rounds, 4 rounds, or 5 rounds of tumor cell stimulation in a re-challenge assay. Each round of stimulation can last for 1 day, 2 days, 3 days, 4 days or 5 days. The persistence of the engineered cells can be measured by the percentage of CD3 positive cells in total live cells.

The percentage of CD3 positive cells in total live cells may be more than 5%, more than 10%, more than 15%, more than 20%, more than 30%, more than 40%, more than 50%, more than 60%, more than 70%, more than 80%, or more than 90% after 1 round, 2 rounds, 3 rounds, 4 rounds, or 5 rounds of stimulation in a re-challenge assay. The percentage of CD3 positive cells in total live cells may be less than 5%, less than 10%, less than 15%, less than 20%, less than 30%, less than 40%, less than 50%, less than 60%, less than 70%, less than 80%, or less than 90% after 1 round, 2 rounds, 3 rounds, 4 rounds, or 5 rounds of stimulation in a re-challenge assay. In some cases, the percentage of CD3 positive cells in total live cells is 10-100%, 10%-50%, 20-100%, 20-60%, 20-40%, 30-70%, 40-80%, 50-90%, 70-100%, 80-100%, or 90-100%, after 1 round, 2 rounds, 3 rounds, 4 rounds, or 5 rounds of stimulation in a re-challenge assay. In some cases, comparing to cells that do not overexpress the YAP peptide or the WWTR1 peptide, the persistence of the engineered cells overexpressing the YAP peptide and/or the WWTR1 peptide is enhanced by more than 5%, more than 10%, more than 15%, more than 20%, more than 30%, more than 40%, more than 50%, more than 60%, more than 70%, more than 80%, more than 90%, more than 100%, more than 150%, more than 200%, more than 250%, more than 300%, more than 400%, more than 500%, more than 600%, more than 700%, more than 800%, more than 900%, or more than 10,00%, after 1 round, 2 rounds, 3 rounds, 4 rounds, or 5 rounds of stimulation in a re-challenge assay. The engineered immune cells may exhibit enhanced long-term persistence than wildtype cells which are not engineered. The amount of engineered immune cells may increase, comparing to control cells, by more than 5%, more than 10%, more than 15%, more than 20%, more than 30%, more than 40%, more than 50%, more than 60%, more than 70%, more than 80%, more than 90%, more than 100%, more than 150%, more than 200%, more than 250%, more than 300%, more than 400%, more than 500%, more than 600%, more than 700%, more than 800%, more than 900%, or more than 10,00%.

The expansion of the engineered cells can be measured by cumulative proliferation fold. In some cases, the engineered cells are amplified by more than 1 fold, more than 2 fold, more than 3 fold, more than 4 fold, more than 5 fold, more than 6 fold, more than 7 fold, more than 8 fold, more than 9 fold, more than 10 fold, more than 20 fold, more than 50 fold, more than 100 fold, more than 200 fold, more than 500 fold, more than 1000 fold, more than 2000 fold, more than 5000 fold, more than 10000 fold, more than 20000 fold, or more than 30000 fold, after 1 round, 2 rounds, 3 rounds, 4 rounds, or 5 rounds of stimulation in a re-challenge assay. In some cases, comparing to cells that do not overexpress the YAP peptide or the WWTR1 peptide, the amount of expansion of the engineered cells overexpressing the YAP peptide and/or the WWTR1 peptide is enhanced by more than 5%, more than 10%, more than 15%, more than 20%, more than 30%, more than 40%, more than 50%, more than 60%, more than 70%, more than 80%, more than 90%, more than 100%, more than 150%, more than 200%, more than 250%, more than 300%, more than 400%, more than 500%, more than 600%, more than 700%, more than 800%, more than 900%, or more than 10,00%, after 1 round, 2 rounds, 3 rounds, 4 rounds, or 5 rounds of stimulation in a re-challenge assay. Comparing to cells that do not overexpress the YAP peptide or the WWTR1 peptide, the engineered cells overexpressing the YAP peptide and/or the WWTR1 peptide may exhibit enhanced long-term persistence.

The exhaustion of the engineered cells can be measured by the expression levels of an exhaustion marker (e.g., PD-1, LAG-3 or TIGIT). In some cases, the percentage of exhaustion marker positive cells is more than 5%, more than 10%, more than 15%, more than 20%, more than 30%, more than 40%, more than 50%, more than 60%, more than 70%, more than 80%, or more than 90% after 1 round, 2 rounds, 3 rounds, 4 rounds, or 5 rounds of stimulation in a re-challenge assay. In some cases, the percentage of exhaustion marker positive cells is less than 5%, less than 10%, less than 15%, less than 20%, less than 30%, less than 40%, less than 50%, less than 60%, less than 70%, less than 80%, or less than 90% after 1 round, 2 rounds, 3 rounds, 4 rounds, or 5 rounds of stimulation in a re-challenge assay. In some cases, the percentage of exhaustion marker positive cells is 10-100%, 10%-50%, 20-100%, 20-60%, 20-40%, 30-70%, 40-80%, 50-90%, 70-100%, 80-100%, or 90-100%, after 1 round, 2 rounds, 3 rounds, 4 rounds, or 5 rounds of stimulation in a re-challenge assay. In some cases, comparing to cells that do not overexpress the YAP peptide or the WWTR1 peptide, the percentage of exhaustion marker positive cells in the engineered cells overexpressing the YAP peptide and/or the WWTR1 peptide is reduced by more than 5%, more than 10%, more than 15%, more than 20%, more than 30%, more than 40%, more than 50%, more than 60%, more than 70%, more than 80%, or more than 90%, after 1 round, 2 rounds, 3 rounds, 4 rounds, or 5 rounds of stimulation in a re-challenge assay.

The engineered cells may migrate into and proliferate in a 3D tumor model. The penetration and persistence of the engineered cells can be measured by the percentage of CD3 positive cells in total live cells (e.g., following the methods described in Example 6). The percentage of CD3 positive cells in total live cells may be more than 5%, more than 10%, more than 15%, more than 20%, more than 30%, more than 40%, more than 50%, more than 60%, more than 70%, more than 80%, or more than 90%. The percentage of CD3 positive cells in total live cells may be less than 5%, less than 10%, less than 15%, less than 20%, less than 30%, less than 40%, less than 50%, less than 60%, less than 70%, less than 80%, or less than 90%. In some cases, the percentage of CD3 positive cells in total live cells is 10-100%, 10%-50%, 20-100%, 20-60%, 20-40%, 30-70%, 40-80%, 50-90%, 70-100%, 80-100%, or 90-100%. In some cases, comparing to cells that do not overexpress the YAP peptide or the WWTR1 peptide, the migration and persistence of the engineered cells overexpressing the YAP peptide and/or the WWTR1 peptide is enhanced by more than 5%, more than 10%, more than 15%, more than 20%, more than 30%, more than 40%, more than 50%, more than 60%, more than 70%, more than 80%, more than 90%, more than 100%, more than 150%, more than 200%, more than 250%, more than 300%, more than 400%, or more than 500%.

The engineered cells may suppress tumor growth in an NCI-H82 mouse xenograft model. The in vivo tumor suppression by the engineered cells can be evaluated by an in vivo tumor suppression assay (e.g., following the methods described in Example 7). NCG mice can be inoculated with NCI-H82 cells subcutaneously to create an NCI-H82 mouse xenograft model. The engineered cells can be administered intravenously to tumor grafted mice 10 days after tumor inoculation. Tumor length (L) and width (W) can be measured by caliper every 3-4 days after CAR-T cells injection. Tumor volume can be estimated using formula: V=(W2×L)/2. The engineered cells may suppress tumor growth by more than 5%, more than 10%, more than 15%, more than 20%, more than 30%, more than 40%, more than 50%, more than 60%, more than 70%, more than 80%, more than 90%, more than 100%, more than 150%, more than 200%, more than 250%, more than 300%, more than 400%, more than 500%, more than 600%, more than 700%, more than 800%, more than 900%, or more than 10,00%, after 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, or 27 days. In some cases, the body weights of the mice do not change significantly throughout the experiment. The body weights of the mice may change by less than 5%, less than 10%, less than 20%, less than 30%, less than 40%, less than 50%, less than 60%, less than 70%, less than 80%, less than 90%, or less than 100%, after 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, or 27 days. In some cases, comparing to cells that do not overexpress the YAP peptide or the WWTR1 peptide, the engineered cells overexpressing the YAP peptide and/or the WWTR1 peptide may have more pronounced tumor suppression. In some cases, comparing to cells that do not overexpress the YAP peptide or the WWTR1 peptide, the tumor inhibition effects of the engineered cells overexpressing the YAP peptide and/or the WWTR1 peptide are enhanced by more than 5%, more than 10%, more than 15%, more than 20%, more than 30%, more than 40%, more than 50%, more than 60%, more than 70%, more than 80%, more than 90%, more than 100%, more than 150%, more than 200%, more than 250%, more than 300%, more than 400%, more than 500%, more than 600%, more than 700%, more than 800%, more than 900%, or more than 10,00%, after 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, or 27 days.

The present disclosure provides a method or process for preparing, manufacturing and/or using the engineered cells for treatment of pathological diseases or conditions.

The cells for engineering can be isolated from a sample, such as a biological sample, e.g., one obtained from or derived from a subject. The subject from which the cell is isolated can be one having the disease or condition or in need of a cell therapy or to which cell therapy will be administered. The subject may be a human in need of a particular therapeutic intervention, such as the adoptive cell therapy for which cells are being isolated, processed, and/or engineered.

The cells may be primary cells, e.g., primary human cells. The samples may include tissue, fluid, and other samples taken directly from the subject, as well as samples resulting from one or more processing steps, such as separation, centrifugation, genetic engineering (e.g., transduction with viral vector), washing, and/or incubation. The biological sample may be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

### Engineered Receptor

One aspect of the present disclosure provides engineered cells (e.g., immune cells) that overexpress the YAP peptide or the WWTR1 peptide described herein and express an engineered receptor. The engineered receptor can comprise an extracellular ligand binding domain or an extracellular antigen binding domain, and optionally an intracellular signaling domain. Exemplary engineered receptors include, but are not limited to, chimeric antigen receptor (CAR), engineered T-cell receptor (TCR), and T-cell antigen coupler (TAC) receptor. The engineered receptor can comprise an extracellular antigen binding domain that specifically binds to an antigen (e.g., a tumor antigen), a transmembrane domain, and an intracellular signaling domain. The intracellular signaling domain can comprise a primary intracellular signaling domain and/or a co-stimulatory signaling domain. The intracellular signaling domain can comprise an intracellular signaling domain of a TCR co-receptor. The engineered receptor can be encoded by a heterologous polynucleotide operably linked to a promoter (such as a constitutive promoter or an inducible promoter).

The engineered receptor can comprise one or more specific binding domains that target at least one tumor antigen, and one or more intracellular effector domains, such as one or more primary intracellular signaling domains and/or co-stimulatory signaling domains.

The engineered receptor can be a chimeric antigen receptor (CAR). Many chimeric antigen receptors are known in the art and can be suitable for the engineered cells described herein. CARs can also be constructed with a specificity for any cell surface marker by utilizing antigen binding fragments or antibody variable domains of, for example, antibody molecules.

CARs of the present disclosure may comprise an extracellular domain comprising at least one antigen binding domain that specifically binds at least one tumor antigen, a transmembrane region, and an intracellular signaling domain.

The intracellular signaling domain may generate a signal that promotes an immune effector function of the CAR-containing cell, e.g., a CAR-T cell. Immune effector function or immune effector response refers to function or response, e.g., of an immune effector cell, that enhances or promotes an immune attack of a target cell. For example, an immune effector function or response can refer to a property of a T or NK cell that promotes killing or the inhibition of growth or proliferation, of a target cell. Examples of immune effector function, e.g., in a CAR-T cell, include cytolytic activity (such as antibody-dependent cellular toxicity, or ADCC) and helper activity (such as the secretion of cytokines). The intracellular signaling domain may generate a signal that promotes proliferation and/or survival of the CAR containing cell. The CAR may comprise one or more intracellular signaling domains selected from the signaling domains of CD28, CD137, CD3, CD27, CD40, ICOS, GITR, and OX40. The signaling domain of a naturally occurring molecule can comprise the entire intracellular or cytoplasmic portion, or the entire native intracellular signaling domain, of the molecule, or a fragment or derivative thereof.

The intracellular signaling domain of a CAR can comprise a primary intracellular signaling domain. “Primary intracellular signaling domain” refers to cytoplasmic signaling sequence that acts in a stimulatory manner to induce immune effector functions. The primary intracellular signaling domain may contain a signaling motif known as Immunoreceptor Tyrosine-based Activation Motif, or ITAM. The primary intracellular signaling domain may comprise a functional signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma R IIa, DAP10, and DAP 12. The primary intracellular signaling domain may comprise a nonfunctional or attenuated signaling domain of a protein selected from the group consisting of CD3 zeta, CD3 gamma, CD3 delta, CD3 epsilon, common FcR gamma (FCER1G), FcR beta (Fc Epsilon Rib), CD79a, CD79b, Fcgamma R IIa, DAP10, and DAP 12. The nonfunctional or attenuated signaling domain can be a mutant signaling domain having a point mutation, insertion or deletion that attenuates or abolishes one or more immune effector functions, such as cytolytic activity or helper activity, including antibody-dependent cellular toxicity (ADCC). The CAR may comprise a nonfunctional or attenuated CD3 zeta (i.e. CD3ζ or CD3z) signaling domain. The intracellular signaling domain may not comprise a primary intracellular signaling domain. An attenuated primary intracellular signaling domain may induce no more than about any of 90%, 80%, 70%, 60%, 50%, 40%, 30%, 20%, 10% or less of an immune effector function (such as cytolytic function against target cells) compared to CARs having the same construct, but with the wild-type primary intracellular signaling domain.

The intracellular signaling domain of a CAR can comprise one or more (such as any of 1, 2, 3, or more) co-stimulatory signaling domains. “Co-stimulatory signaling domain” can be the intracellular portion of a co-stimulatory molecule. The term “co-stimulatory molecule” refers to a cognate binding partner on an immune cell (such as T cell) that specifically binds with a co-stimulatory ligand, thereby mediating a co-stimulatory response by the immune cell, such as, but not limited to, proliferation and survival. Co-stimulatory molecules are cell surface molecules other than antigen receptors or their ligands that contribute to an efficient immune response. A co-stimulatory molecule can be represented in the following protein families: TNF receptor proteins, Immunoglobulin-like proteins, cytokine receptors, integrins, signaling lymphocytic activation molecules (SLAM proteins), and activating NK cell receptors. Co-stimulatory molecules include, but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such co-stimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CDIOO (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

The CAR can comprise a single co-stimulatory signaling domain. The CAR can comprise two or more co-stimulatory signaling domains. The intracellular signaling domain can comprise a functional primary intracellular signaling domain and one or more co-stimulatory signaling domains. The CAR may not comprise a functional primary intracellular signaling domain (such as CD3ζ). The CAR can comprise an intracellular signaling domain consisting of or consisting essentially of one or more co-stimulatory signaling domains. The CAR can comprise an intracellular signaling domain consisting of or consisting essentially of a nonfunctional or attenuated primary intracellular signaling domain (such as a mutant CD3ζ) and one or more co-stimulatory signaling domains. Upon binding of the antigen binding domain to tumor antigen, the co-stimulatory signaling domains of the CAR can transduce signals for enhanced proliferation, survival and differentiation of the modified immune cells having the CAR (such as T cells), and inhibit activation induced cell death. The one or more co-stimulatory signaling domains can be derived from one or more molecules selected from the group consisting of CD27, CD28, 4-1BB (i.e., CD137), OX40, CD30, CD40, CD3, lymphocyte function-associated antigen-1 (LFA-1), CD2, CD7, LIGHT, NKG2C, B7-H3 and ligands that specially bind to CD83.

The intracellular signaling domain of a CAR can comprise a co-stimulatory signaling domain derived from CD28. The intracellular signaling domain can comprise a primary intracellular signaling domain of CD30 and a co-stimulatory signaling domain of CD28. The intracellular signaling domain in the chimeric receptor of the present application can comprise a co-stimulatory signaling domain derived from 4-1BB (i.e., CD137). The intracellular signaling domain can comprise a primary intracellular signaling domain of CD3ζ and a co-stimulatory signaling domain of 4-1BB.

The intracellular signaling domain of the CAR can comprise a co-stimulatory signaling domain of CD28 and a co-stimulatory signaling domain of 4-1BB. The intracellular signaling domain can comprise a primary intracellular signaling domain of CD3ζ, a co-stimulatory signaling domain of CD28, and a co-stimulatory signaling domain of 4-1BB. The intracellular signaling domain can comprise a polypeptide comprising from the N-terminus to the C-terminus: a co-stimulatory signaling domain of CD28, a co-stimulatory signaling domain of 4-1BB, and a primary intracellular signaling domain of CD3ζ.

The antigen binding domain of a CAR may comprise one or more (such as any one of 1, 2, 3, 4, 5, 6 or more) antibodies or antibody fragments, which can be selected from an scFv, a Fv, a Fab, a (Fab′)₂, a minibody, a diabody, a single domain antibody (sdAb), or a VHH domain. The antigen binding domain of a CAR can comprise a ligand or an extracellular portion of a receptor that specifically binds to a tumor antigen. The CAR can be a monospecific, bispecific or multispecific CAR. The antigen binding domain of a CAR can specifically bind to a single tumor antigen. The antigen binding domain of a CAR can bind to two or more tumor antigens. The engineered receptor (e.g., CAR) may redirect the specificity of the engineered cells through the expression of a chimeric antigen receptor (CAR) or TCR on these cells.

The antigen may be a tumor antigen selected from the group consisting of BCMA, CLL1, CD4, GPC3, GPRC5D, GU2CYC, CD19, MUC16, MUC1, CAIX, CEA, CD8, CD7, CD10, CD20, CD22, CD30, CD33, CD34, CD38, CD41, CD44, CD49f, CD56, CD74, CD133, CD138, EGP-2, EGP-40, EpCAM, ERBB2, ERBB3, ERBB4, FBP, fetal acetylcholine receptor, folate receptor-α, GD2, GD3, hTERT, IL-13Rα2, κ-light chain, KDR, LeY, L1 cell adhesion molecule, MAGE-A1, mesothelin, MAGEA3, p53, MART1, GP100, proteinase-3 (PR3), tyrosinase, survivin, hTERT, EphA2, NY-ESO-1, h5T4, PSCA, PSMA, ROR1, TAG-72, VEGF-R2, WT-1, CD123, CD44V6, NKCS1, IGF1R, EGFR, EGFR-VIII, Claudin 18.2, Claudin 6, NKG2D, Delta-like 3 (DLL3), CD70, CS-1, c-Met, Glycolipid F77, PD-L1, PD-L2, and other tumor antigens with clinical significance, and combinations thereof. The antigen may be DLL3.

The tumor antigen can be derived from an intracellular protein of tumor cells. The tumor antigen can be expressed on the surface of tumor cells. Many TCRs specific for tumor antigens (including tumor-associated antigens) have been described, including, for example, NY-ESO-1 cancer-testis antigen, the p53 tumor suppressor antigens, TCRs for tumor antigens in melanoma (e.g., MART1, gp 100), leukemia (e.g., WT1, minor histocompatibility antigens), and breast cancer (e.g., HER2, NY-BR1).

The transmembrane region of a CAR comprises a transmembrane region can be selected from the transmembrane region of an alpha, beta or zeta chain of a T-cell receptor, CD28, CD3 epsilon, CD45, CD4, CD5, CD8, CD9, CD16, CD22, CD33, CD37, CD64, CD80, CD86, CD134, CD137, CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL-2R beta, IL-2R gamma, IL-7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRT AM, Ly9 (CD229), CD160 (BY55), PSGL1, CDIOO (SEMA4D), SLAMF6 (NTB-A, Ly108), SLAM (SLAMF1, CD150, IPO-3), BLAME (SLAMF8), SELPLG (CD162), LTBR, PAG/Cbp, NKp44, NKp30, NKp46, NKG2D, and/or NKG2C. The transmembrane domain of the CAR can be a CD4, CD3, CD8α, or CD28 transmembrane domain. The transmembrane region of the CAR can comprise a CD8α transmembrane domain.

The extracellular domain can be connected to the transmembrane domain by a hinge domain. The hinge domain can be a hinge domain of CD8a.

The CAR can also comprise a signal peptide (SP), such as a CD8α signal peptide. The signal peptide can comprise a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 5.

Many CARs targeting different tumor antigens have been widely disclosed in the field, such as CD19 CARs or BCMA CARs. The extracellular antigen binding domain of CD19 CARs can be or include the CD19 binding fragment (e.g., FMC63, SJ25C1, or those disclosed in different patents such as WO 2022/012683, etc). BCMA CARs also have been well described, related patents include but not limited to WO 2016/014789, WO 2016/014565, WO 2013/154760, and WO 2018/028647, etc. The extracellular antigen binding domain of BCMA CARs may be or include BCMA binding fragment. The BCMA binding fragment may bind to one or more epitopes on BCMA. The BCMA CARs may be bivalent CARs comprising two anti-BCMA sdAbs targeting same or different BCMA epitopes.

The CAR can be a DLL3 CAR. The DLL3 CAR may be monospecific. The DLL3 CAR may be bispecific or bivalent. The extracellular antigen binding domain of DLL3 CAR may be or include one or more DLL3 binding moiety. The DLL3 binding moiety may bind to one or more epitopes on DLL3. The DLL3 CAR may be bivalent CARs comprising two anti-DLL3 sdAbs (e.g., VHH domains) targeting same or different DLL3 epitopes. A wide variety of antigen binding domain sequences can be used as the antigen binding domain of the DLL3 CAR (e.g., AMG119 and AMG157, and those disclosed in different patents such as WO2019200007A1, WO2020180591A1, WO2021008610A1, etc).

The DLL3 CAR can comprise from the N-terminus to the C-terminus: a CD8α signal peptide, an extracellular antigen binding domain comprising two anti-DLL3 sdAbs (e.g., VHH domains), a CD8α hinge domain, a CD8α transmembrane domain, a 4-1BB co-stimulatory signaling domain, and a CD3ζ primary intracellular signaling domain. The anti-DLL3 sdAb (e.g., VHH domain) can comprise a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 6 or SEQ ID NO: 7.

The CD8α hinge domain can comprise a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 8.

The CD8α transmembrane domain can comprise a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 9.

The 4-1BB co-stimulatory signaling domain can comprise a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 10.

The CD3ζ primary intracellular signaling domain can comprise a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 11.

The DLL3 CAR can comprise a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 12. The DLL3 CAR may specifically bind to DLL3-positive tumor cells (e.g., NCI-H82 cells).

The engineered receptor can be a modified T-cell receptor or engineered T-cell receptor. The engineered TCR can be specific for a tumor antigen. The tumor antigen can be selected from the group consisting of BCMA, CLL1, CD4, GPC3, GPRC5D, GU2CYC, CD19, MUC16, MUC1, CAIX, CEA, CD8, CD7, CD10, CD20, CD22, CD30, CD33, CD3ζ, CD38, CD41, CD44, CD49f, CD56, CD74, CD133, CD138, EGP-2, EGP-40, EpCAM, ERBB2, ERBB3, ERBB4, FBP, fetal acetylcholine receptor, folate receptor-α, GD2, GD3, HER-2, hTERT, IL-13R-α2, κ-light chain, KDR, LeY, L1 cell adhesion molecule, MAGE-A1, mesothelin, MAGEA3, p53, MART1, GP100, proteinase-3 (PR3), tyrosinase, survivin, hTERT, EphA2, NY-ESO-1, h5T4, PSCA, PSMA, ROR1, TAG-72, VEGF-R2, WT-1, CD123, CD44V6, NKCS1, IGF1R, EGFR, EGFR-VIII, Claudin 18.2, Claudin 6, NKG2D, Delta-like 3 (DLL3), CD70, CS-1, c-Met, Glycolipid F77, PD-L1, PD-L2, and other tumor antigens with clinical significance, and combinations thereof. The tumor antigen can be derived from an intracellular protein of tumor cells. The tumor antigen can be expressed on the surface of tumor cells. Many TCRs specific for tumor antigens (including tumor-associated antigens) have been described, including, for example, NY-ESO-1 cancer-testis antigen, the p53 tumor suppressor antigens, TCRs for tumor antigens in melanoma (e.g., MART1, gp 100), leukemia (e.g., WT1, minor histocompatibility antigens), and breast cancer (e.g., HER2, NY-BR1). Any of the TCRs known in the art can be used. The TCR can have an enhanced affinity to the tumor antigen. Exemplary TCRs and methods for introducing the TCRs to immune cells have been described, for example, in U.S. Pat. No. 5,830,755, and Kessels et al. Immunotherapy through TCR gene transfer. Nat. Immunol. 2, 957-961 (2001), which are incorporated herein by reference in the entirety.

The TCR receptor complex is an octomeric complex formed by variable TCR receptor α and β chains (or γ and δ chains on case of γδ T cells) with three dimeric signaling modules CD3δ/ε, CD3γ/ε and CD247 (T-cell surface glycoprotein CD3 zeta chain) ζ/ζ or ζ/η. Ionizable residues in the transmembrane region of each subunit form a polar network of interactions that hold the complex together. TCR complex has the function of activating signaling cascades in T cells.

The engineered receptor can be an engineered TCR comprising one or more T-cell receptor (TCR) fusion proteins (TFPs). Exemplary TFPs have been described, for example, in US20170166622A1, which is incorporated herein by reference in its entirety. The TFP can comprise an extracellular domain of a TCR subunit that comprises an extracellular domain or portion thereof of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. The TFP can comprise a transmembrane region that comprises a transmembrane region of a protein selected from the group consisting of a TCR alpha chain, a TCR beta chain, a CD3 epsilon TCR subunit, a CD3 gamma TCR subunit, a CD3 delta TCR subunit, functional fragments thereof, and amino acid sequences thereof having at least one but not more than 20 modifications. The TFP can comprise a transmembrane region that comprises a transmembrane region of a protein selected from the group consisting of a TCR alpha chain, a TCR beta chain, a TCR zeta chain, a CD3 epsilon TCR subunit, a CD3 gamma TCR subunit, a CD3 delta TCR subunit, CD45, CD4, CD5, CD8, CD9, CD16, CD22, CD33, CD28, CD37, CD64, CD80, CD86, CD134, CD137, CD154, functional fragments thereof, and amino acid sequences thereof having at least one but not more than 20 modifications.

The TFP can comprise a TCR subunit comprising at least a portion of a TCR extracellular domain, and a TCR intracellular domain comprising a stimulatory domain from an intracellular signaling domain of CD3 epsilon; and an antigen binding domain, wherein the TCR subunit and the antigen binding domain are operatively linked, and wherein the TFP incorporates into a TCR when expressed in a T cell.

The engineered receptor can be a T-cell antigen coupler (TAC) receptor. Exemplary TAC receptors have been described, for example, in US20160368964A1, which is incorporated herein by reference. The TAC can comprise an antigen binding domain, a TCR-binding domain that specifically binds a protein associated with the TCR complex, and a T-cell receptor signaling domain. The antigen binding domain can be an antibody fragment, such as scFv or VHH, which specifically binds to a tumor antigen. The antigen binding domain can be a designed Ankyrin repeat (DARPin) polypeptide. The tumor antigen can be selected from the group consisting of BCMA, CLL1, CD4, GPC3, GPRC5D, GU2CYC, CD19, MUC16, MUC1, CAIX, CEA, CD8, CD7, CD10, CD20, CD22, CD30, CD33, CD34, CD38, CD41, CD44, CD49f, CD56, CD74, CD133, CD138, EGP-2, EGP-40, EpCAM, ERBB2, ERBB3, ERBB4, FBP, fetal acetylcholine receptor, folate receptor-α, GD2, GD3, HER-2, hTERT, IL-13R-α2, κ-light chain, KDR, LeY, L1 cell adhesion molecule, MAGE-A1, mesothelin, MAGEA3, p53, MART1, GP100, proteinase-3 (PR3), tyrosinase, survivin, hTERT, EphA2, NY-ESO-1, h5T4, PSCA, PSMA, ROR1, TAG-72, VEGF-R2, WT-1, CD123, CD44V6, NKCS1, IGF1R, EGFR, EGFR-VIII, Claudin 18.2, Claudin 6, NKG2D, Delta-like 3 (DLL3), CD70, CS-1, c-Met, Glycolipid F77, PD-L1, PD-L2, and other tumor antigens with clinical significance, and combinations thereof. The tumor antigen can be derived from an intracellular protein of tumor cells. The tumor antigen can be expressed on the surface of tumor cells. The protein associated with the TCR complex can be CD3, such as CD3E. The TCR-binding domain can be a single chain antibody, such as scFv, or a V_{H}H. The TCR-binding domain can be derived from UCHT1. The TAC receptor can comprise a cytosolic domain and a transmembrane region. The T-cell receptor signaling domain can comprise a cytosolic domain derived from a TCR co-receptor. Exemplary TCR co-receptors include, but are not limited to, CD4, CD8, CD28, CD45, CD4, CD5, CD9, CD16, CD22, CD33, CD37, CD64, CD80, CD86, CD134, CD137 and CD 154. The TAC receptor can comprise a transmembrane region and a cytosolic domain derived from CD4. The TAC receptor can comprise a transmembrane region and a cytosolic domain derived from CD8 (such as CD8a).

T cell co-receptors are expressed as membrane proteins on T cells. They can provide stabilization of the TCR: peptide: MEC complex and facilitate signal transduction. The two subtypes of T cell co-receptor, CD4 and CD8, display strong specificity for particular MEC classes. The CD4 co-receptor can only stabilize TCR: MEC II complexes while the CD8 co-receptor can only stabilize the TCR: MEC I complex. The differential expression of CD4 and CD8 on different T cell types results in distinct T cell functional subpopulations. CD8+ T cells are cytotoxic T cells.

The engineered receptor (such as CAR, TCR, or TAC) can target one or more tumor antigens. Tumor antigens are proteins that are produced by tumor cells that can elicit an immune response, particularly T-cell mediated immune responses. The selection of the targeted antigen will depend on the particular type of cancer to be treated. Exemplary tumor antigens include, for example, a glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-la, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin.

The tumor antigen can comprise one or more antigenic cancer epitopes associated with a malignant tumor. Malignant tumors express a number of proteins that can serve as target antigens for an immune attack. These molecules include but are not limited to tissue-specific antigens such as MART-1, tyrosinase and gp100 in melanoma and prostatic acid phosphatase (PAP) and prostate-specific antigen (PSA) in prostate cancer. Other target molecules belong to the group of transformation-related molecules such as the oncogene HER2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B cell differentiation antigens such as CD 19, CD20 and CD37 are other candidates for target antigens in B-cell lymphoma.

The tumor antigen can be a tumor-specific antigen (TSA) or a tumor-associated antigen (TAA). A TSA is unique to tumor cells and does not occur on other cells in the body. A TAA associated antigen is not unique to a tumor cell, and instead is also expressed on a normal cell under conditions that fail to induce a state of immunologic tolerance to the antigen. The expression of the antigen on the tumor can occur under conditions that enable the immune system to respond to the antigen. TAAs can be antigens that are expressed on normal cells during fetal development, when the immune system is immature, and unable to respond or they can be antigens that are normally present at extremely low levels on normal cells, but which are expressed at much higher levels on tumor cells.

Non-limiting examples of TSA or TAA antigens include the following: Differentiation antigens such as MART-1/MelanA (MART-I), gp 100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23HI, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS 1, SDCCAG16, TA-90\Mac-2 binding protein\cyclophilin C-associated protein, TAAL6, TAG72, TLP, and TPS.

### Nucleic Acids

The present disclosure provides (i) nucleic acids encoding the YAP peptide and/or the WWTR1 peptide, and (ii) nucleic acids encoding an engineered receptor (e.g., CAR, TAC or TCR). The nucleic acids of the present disclosure can comprise a nucleic acid sequence encoding any one of the YAP peptide, the WWTR1 peptide, CARs, and/or TCRs disclosed herein. The nucleic acid can encode both (1) a CAR and (2) a YAP peptide or a WWTR1 peptide. The nucleic acid may comprise one, two, three, four, five or more copies of the coding sequence of the YAP peptide and/or the WWTR1 peptide. The expression of the YAP peptide and/or the WWTR1 peptide may be controlled by a non-native regulatory element.

A nucleic acid of the present disclosure may comprise a first nucleic acid sequence and a second nucleic acid sequence. The first nucleic acid may be upstream of the second nucleic acid or the first nucleic acid may be downstream of the second nucleic acid. The first and second nucleic acid sequence can be separated by a linker. A linker for use in the present disclosure allows for multiple proteins to be encoded by the same nucleic acid sequence (e.g., a multicistronic or bicistronic sequence), which are translated as a polyprotein that is dissociated into separate protein components. The nucleic acid may comprise from 5′ end to 3′ end of the first nucleic acid sequence, the linker, and the second nucleic acid sequence. The nucleic acid may comprise from 5′ end to 3′ end the second nucleic acid sequence, the linker, and the first nucleic acid sequence. The first nucleic acid sequence may encode a first CAR described herein and the second nucleic acid sequence may encode the YAP peptide and/or the WWTR1 peptide described herein.

The linker may comprise a nucleic acid sequence that encodes for an internal ribosome entry site (IRES). As used herein, “an internal ribosome entry site” or “IRES” refers to an element that promotes direct internal ribosome entry to the initiation codon, such as ATG, of a protein coding region, thereby leading to cap-independent translation of the gene. Various internal ribosome entry sites are known to those of skill in the art, including, without limitation, IRES obtainable from viral or cellular mRNA sources, e.g., immunogloublin heavy-chain binding protein (BiP); vascular endothelial growth factor (VEGF); fibroblast growth factor 2; insulin-like growth factor; translational initiation factor eIF4G; yeast transcription factors TFIID and HAP4; and IRES obtainable from, e.g., cardiovirus, rhinovirus, aphthovirus, HCV, Friend murine leukemia virus (FrMLV), and Moloney murine leukemia virus (MoMLV). Those of skill in the art would be able to select the appropriate IRES.

The linker may comprise a nucleic acid sequence that encodes for a self-cleaving peptide. As used herein, a “self-cleaving peptide” or “2A linker” refers to an oligopeptide that allow multiple proteins to be encoded as polyproteins, which dissociate into component proteins upon translation. Use of the term “self-cleaving” is not intended to imply a proteolytic cleavage reaction. Various self-cleaving or 2A linkers are known to those of skill in the art, including, without limitation, those found in members of the Picornaviridae virus family, e.g., foot-and-mouth disease virus (FMDV), equine rhinitis A virus (ERAV0, Thosea asigna virus (TaV), and porcine tescho virus-1 (PTV-1); and carioviruses such as Theilovirus and encephalomyocarditis viruses. 2A linkers derived from FMDV, ERAV, PTV-1, and TaV are referred to herein as “F2A,” “E2A,” “P2A,” and “T2A,” respectively. The P2A linker may have a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 17. The T2A linker may have a sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 18.

Various linker sequences are known in the art, including, without limitation, glycine serine (GS) spacers (also known as GS linkers) such as (GS)n, (SG)n, (GSGGS)n and (GGGS)n, where n represents an integer of at least 1. Exemplary linker sequences can comprise an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence of SEQ ID NO: 19, SEQ ID NO: 20, SEQ ID NO: 21, SEQ ID NO: 22, SEQ ID NO: 23, SEQ ID NO: 24, or SEQ ID NO: 25.

A nucleic acid of the present disclosure may a restriction enzyme site sequence.

A nucleic acid of the present disclosure can be operably linked to a transcriptional control element, e.g., a promoter, and enhancer, etc.

The promoter may be a CD8 cell-specific promoter, a CD4 cell-specific promoter, a neutrophil-specific promoter, or an NK-specific promoter. For example, a CD4 gene promoter can be used; see, e.g., Salmon et al. Proc. Natl. Acad. Sci. USA (1993) 90:7739; and Marodon et al. (2003) Blood 101:3416. As another example, a CD8 gene promoter can be used. NK cell-specific expression can be achieved by use of an NcrI (p46) promoter; see, e.g., Eckelhart et al. Blood (2011) 117:1565.

Other examples of suitable promoters include the immediate early cytomegalovirus (CMV) promoter sequence. This promoter sequence is a strong constitutive promoter sequence capable of driving high levels of expression of any nucleic acid sequence operatively linked thereto. Other constitutive promoter sequences can also be used, including, but not limited to a simian virus 40 (SV40) early promoter, a mouse mammary tumor virus (MMTV) or human immunodeficiency virus (HIV) long terminal repeat (LTR) promoter, a MoMuLV promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the disclosure should not be limited to the use of constitutive promoters. Inducible promoters are also contemplated as part of the disclosure. The use of an inducible promoter provides a molecular switch capable of turning on expression of the nucleic acid sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The nucleic acid of the present disclosure may be provided for the production of (i) a YAP peptide and/or a WWTR1 peptide described herein, and/or (ii) a CAR described herein (e.g., in a mammalian cell). The nucleic acid of the present disclosure may provide for amplification of the nucleic acid.

A vector, for example an expression vector (e.g., a lentiviral vector) can be used to introduce the nucleic acid described herein into an immune cell (e.g., a T cell) or precursor thereof. The vector (e.g., a lentiviral vector) of the present disclosure may comprise one or more nucleic acids encoding for the YAP peptide and/or the WWTR1 peptide described herein. The vector (e.g., lentiviral vector) may comprise additional elements that will aid in the functional expression of the YAP peptide and/or the WWTR1 peptide described herein, and/or the CAR described herein. The expression vector may comprise a mammalian promoter. The vector may comprise an elongation-factor-1-alpha promoter (EF-1α promoter). The use of an EF-1a promoter may increase the efficiency in expression of downstream transgenes (e.g., a CAR encoding nucleic acid). Physiologic promoters (e.g., an EF-1α promoter) may be less likely to induce integration mediated genotoxicity, and may abrogate the ability of the retroviral vector to transform stem cells. Other physiological promoters suitable for use in a vector (e.g., lentiviral vector) can be incorporated into a vector of the present disclosure. The vector (e.g., lentiviral vector) may comprise a non-requisite cis acting sequence that can improve titers and gene expression.

The nucleic acid can encode a naked CAR. The nucleic acid can comprise from the 5′ end to the 3′ end, a CD8α signal peptide, an extracellular antigen binding domain, a CD8α hinge domain, a CD8α transmembrane domain, a CD137 co-stimulatory signaling domain, a CD3ζ primary intracellular signaling domain. In some cases, the nucleic acid encodes an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in SEQ ID NO: 12.

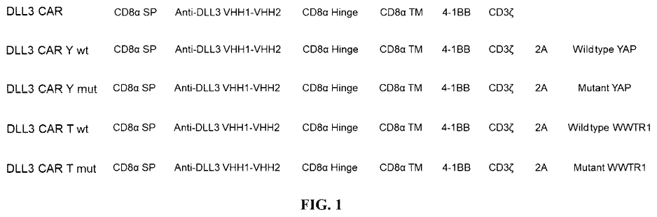

The nucleic acid can encode (1) a CAR and (2) a YAP peptide and/or a WWTR1 peptide. The nucleic acid can comprise from the 5′ end to the 3′ end, the coding sequences of a YAP peptide or a WWTR1 peptide, a 2A cleavable linker, and a CAR. The nucleic acid can comprise from the 5′ end to the 3′ end, the coding sequences of a CAR, a 2A cleavable linker, and a YAP peptide or a WWTR1 peptide. In some cases, nucleic acid encodes an amino acid sequence that is at least 70%, 80%, 81%, 82%, 83%, 84%, 85%, 86%, 87%, 88%, 89%, 90%, 91%, 92%, 93%, 94%, 95%, 96%, 97%, 98%, 99%, or 100% identical to the amino acid sequence set forth in SEQ ID NO: 13, SEQ ID NO: 14, SEQ ID NO: 15, or SEQ ID NO: 16. Exemplary structures of the nucleic acids described herein are shown in FIG. 1.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and may be at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

### Methods of Treatment

The engineered cells (e.g., engineered immune cells) with enhanced TEAD transcriptional activities, particularly the engineered cells overexpressing the YAP peptide and/or the WWTR1 peptide described herein, can be used in a variety of experimental, therapeutic and commercial applications.

In one aspect, the disclosure provides a pharmaceutical composition comprising the engineered cell described herein and a pharmaceutically acceptable carrier.

In one aspect, the disclosure provides a method of treating a disease or disorder in a subject (e.g., human subject), the method comprising administering to the subject, an effective amount of the engineered cell described herein, or the pharmaceutical composition described herein. The disease or disorder may be cancer, an autoimmune disease, or an infection.

The disease or disorder may be solid tumor. “Solid tumor” refers to an abnormal mass of tissue that usually does not contain cysts or liquid areas. Solid tumors may be benign (not cancer), or malignant (cancer). Different types of solid tumors are named for the type of cells that form them. Examples of solid tumors are sarcomas, carcinomas, and lymphomas. Leukemias (cancers of the blood) generally do not form solid tumors.

In one aspect, the disclosure provides a method of modulating an immune response comprising administering an effective amount of engineered cells described herein to a subject in need thereof.

The term “effective amount” as used herein means an amount effective, at dosages and for periods of time necessary to achieve the desired results.

In another aspect, the present disclosure provides a method for treating cancer comprising administering an effective amount of engineered cells described herein to a subject in need thereof. Examples of cancer that can be treated include, but are not limited to, small cell lung cancer (SCLC), large cell neuroendocrine cancer (LCNC), neuroendocrine prostate cancer (NEPC), pancreatic neuroendocrine tumor (PNET), gastrointestinal neuroendocrine cancers, leukemias including chronic lymphocytic leukemia, chronic myelogenous leukemia, acute myelogenous leukemia, acute lymphoblastic leukemia, and T cell and B cell leukemias, lymphomas (Hodgkin's and non-Hodgkins), lymphoproliferative disorders, plasmacytomas, histiocytomas, melanomas, adenomas, sarcomas, carcinomas of solid tissues, hypoxic tumors, squamous cell carcinomas, genitourinary cancers such as cervical and bladder cancer, hematopoietic cancers, head and neck cancers, and nervous system cancers.

The disclosure further includes the use of the engineered cells described herein in the manufacture of a medicament or pharmaceutical composition to modulate an immune response, to treat an infection or to treat cancer as described hereinabove.

The engineered cells can also be used in experimental models, for example, to further study and elucidate the function of the cells.

One or more of the engineered cells described herein can be administered to a subject in a single, unified form, such as an intravenous injection, or in multiple forms, for example, as multiple intravenous infusions or injections, or subcutaneous injections. In some cases, the engineered cells can expand within a subject's body, in vivo, after administration to a subject. The engineered cells can be frozen to provide cells for multiple treatments with the same cell preparation. The engineered cells of the disclosure, and pharmaceutical compositions comprising the same, can be packaged as a kit. A kit can include instructions (e.g., written instructions) on the use of the engineered cells and compositions comprising the same.

A method of treatment can comprise administering to a subject a therapeutically-effective amount of the engineered cells. The therapeutically-effective amount of the engineered cells may be administered for at least 1 week, 2 weeks, 3 weeks, 1 month, 2 months, 3 months, 4 months, 5 months, 6 months, or 1 year. The therapeutically-effective amount of the engineered cells may be administered for at least one week. The therapeutically-effective amount of the engineered cells may be administered for at least two weeks.

The engineered cells described herein can be administered before, during, or after the occurrence of a disease or condition, and the timing of administering the engineered cells can vary. For example, the engineered cells can be used as a prophylactic and can be administered continuously to subjects with a propensity to conditions or diseases in order to lessen a likelihood of the occurrence of the disease or condition. The engineered cells can be administered to a subject during or as soon as possible after the onset of the symptoms. The administration of the engineered cells can be initiated immediately within the onset of symptoms, within the first 3 hours of the onset of the symptoms, within the first 6 hours of the onset of the symptoms, within the first 24 hours of the onset of the symptoms, within 48 hours of the onset of the symptoms, or within any period of time from the onset of symptoms. The initial administration can be via any route practical (e.g., intravenous infusions or injections), such as by any route described herein using any formulation described herein. In some examples, the administration of the engineered cells of the disclosure is an intravenous administration. One or multiple dosages of the engineered cells can be administered as soon as is practicable after the onset of a cancer or an infectious disease, and for a length of time necessary for the treatment of the disease, such as, for example, from about 24 hours to about 48 hours, from about 48 hours to about 1 week, from about 1 week to about 2 weeks, from about 2 weeks to about 1 month, from about 1 month to about 3 months. For the treatment of cancer, one or multiple dosages of the engineered cells can be administered years after onset of the cancer and before or after other treatments. In some examples, the engineered cells can be administered for at least about 10 minutes, 30 minutes, 1 hour, 2 hours, 3 hours, 4 hours, 5 hours, 6 hours, 12 hours, 24 hours, at least 48 hours, at least 72 hours, at least 96 hours, at least 1 week, at least 2 weeks, at least 3 weeks, at least 4 weeks, at least 1 month, at least 2 months, at least 3 months, at least 4 months, at least 5 months, at least 6 months, at least 7 months, at least 8 months, at least 9 months, at least 10 months, at least 11 months, at least 12 months, at least 1 year, at least 2 years at least 3 years, at least 4 years, or at least 5 years. The length of treatment can vary for each subject.

Methods for administration of engineered cells for adoptive cell therapy are known and can be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8 (10): 577-85). See, e.g., Themeli et al. (2013) Nat Biotechnol. 31 (10): 928-933; Tsukahara et al. (2013) Biochem Biophys Res Commun 438 (1): 84-9; Davila et al. (2013) PLOS ONE 8 (4): e61338. The cell therapy, e.g., adoptive T cell therapy can be carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

The cell therapy (e.g., adoptive T cell therapy) can be carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. The first and second subjects may be genetically identical. The first and second subjects may be genetically similar. The second subject may express the same HLA class or supertype as the first subject.

The subject (e.g., human subject) may have been treated with a therapeutic agent targeting the disease or condition, e.g., the tumor, prior to administration of the cells or composition containing the cells. The subject may be refractory or non-responsive to the other therapeutic agent. The subject may have persistent or relapsed disease, e.g., following treatment with another therapeutic intervention, including chemotherapy, radiation, and/or hematopoietic stem cell transplantation (HSCT), e.g., allogenic HSCT. In some cases, the administration can effectively treat the subject despite the subject having become resistant to another therapy.

The subject may be responsive to the other therapeutic agent, and treatment with the therapeutic agent reduces disease burden. The subject may be initially responsive to the therapeutic agent, but exhibits a relapse of the disease or condition over time. The subject may have not relapsed. The subject may be determined to be at risk for relapse, such as at a high risk of relapse, and thus the cells are administered prophylactically, e.g., to reduce the likelihood of or prevent relapse. The subject may has not received prior treatment with another therapeutic agent.

The subject may have persistent or relapsed disease, e.g., following treatment with another therapeutic intervention, including chemotherapy, radiation, and/or hematopoietic stem cell transplantation (HSCT), e.g., allogenic HSCT. The administration may effectively treat the subject despite the subject having become resistant to another therapy.

The engineered cells described herein can be administered to an animal, such as a mammal, even more a human, to treat a cancer. In addition, the engineered cells can be used for the treatment of any condition related to a cancer, especially a cell-mediated immune response against a tumor cell(s), where it is desirable to treat or alleviate the disease.

The engineered cells (e.g., immune cells, T cells, or NK cells) described herein can be included in a composition for immunotherapy. The composition can include a pharmaceutical composition and further include a pharmaceutically acceptable carrier. A therapeutically effective amount of the pharmaceutical composition comprising the engineered cells can be administered.

The engineered cells can be immediately used in the above therapeutic, experimental or commercial applications or the cells can be cryopreserved for use at a later date. The pharmaceutical compositions can be included in a container, pack, or dispenser together with instructions for administration.

The engineered cells disclosed herein can be formulated in unit dosage forms suitable for single administration of precise dosages. The unit dosage forms may comprise additional lymphocytes. In unit dosage form, the formulation is divided into unit doses containing appropriate quantities of one or more compounds. The unit dosage can be in the form of a package containing discrete quantities of the formulation. Non-limiting examples are packaged tablets or capsules, and powders in vials or ampoules. Aqueous suspension compositions can be packaged in single-dose non-reclosable containers. Multiple-dose reclosable containers can be used, for example, in combination with a preservative or without a preservative. The pharmaceutical composition may not comprise a preservative. Formulations for parenteral injection can be presented in unit dosage form, for example, in ampoules, or in multi-dose containers with a preservative.

### EXAMPLES

The disclosure is further described in the following examples, which do not limit the scope of the disclosure described in the claims. In this disclosure, pro-inflammatory SCLC transcription factors or TEAD coactivators (e.g., a YAP peptide or a WWTR1 peptide) were employed to deliver transcriptional inputs to drive the anti-tumor activity of adoptive transferred cells, such as CAR T cells. DLL3 CAR T cells overexpressing YAP and/or WWTR1 are used as proof-of-concept examples to demonstrate the superiority of YAP armored CAR T cells and WWTR1 armored CAR T cells in comparison to conventional CAR T cells.

### Example 1. Plasmid Construction, Virus Preparation, and Titer Evaluation

The lentiviral vector pLVX-Puro (Clontech #632164) was modified by replacing the original promoter with human elongation factor 1α promoter (hEF1α) and by removing the puromycin resistance gene with EcoRI and BamHI. PLVX-EF1A was further subjected to a lentivirus packaging procedure as described below.

To generate viral particles comprising nucleic acids encoding the CAR constructs disclosed herein, lentivirus packaging plasmid mixture including pMDLg/pRRE (Addgene #11251), pRSV-Rev (Addgene #11253), and pMD2.G (Addgene #11259) were pre-mixed with a PLVX-EFIA (including target system) vector at a pre-optimized ratio with polyetherimide (PEI), mixed properly, and incubated at room temperature for 5 min. The transfection mix was added dropwise to HEK293-T cells and mixed gently. Transfected HEK293-T cells were incubated overnight at 37° C. and 5% CO₂. Twenty-four hours post-transfection, supernatants were collected and centrifuged at 4° C., 500 g for 10 min to remove any cellular debris. Centrifuged supernatants were filtered through a 0.45 μm PES filter to concentrate the viral supernatants before ultracentrifugation. After ultracentrifugation, the supernatant was carefully discarded and the virus pellets were rinsed with pre-chilled DPBS. The concentration of virus particles was measured. Virus particles were aliquoted and stored at −80° C. Viral titers are determined by functional transduction on a T cell line.

### Example 2. Preparation of Engineered T Cells Expressing Chimeric Antigen Receptors

The constructs of DLL3 VHH CAR (illustrated as DLL3 CAR, SEQ ID NO: 12), DLL3 CAR armored with wildtype YAP (illustrated as DLL3 CAR Y wt, SEQ ID NO: 13), DLL3 CAR armored with mutant YAP (illustrated as DLL3 CAR Y mut, SEQ ID NO: 14), DLL3 CAR armored with wildtype WWTR1 (illustrated as DLL3 CAR T wt, SEQ ID NO: 15) and DLL3 CAR armored with mutant WWTR1 (illustrated as DLL3 CAR T mut, SEQ ID NO: 16) were designed as shown in FIG. 1. The nucleotide sequences of CAR backbones encoding DLL3 CAR, YAP and/or WWTR1 polypeptides were chemically synthesized and cloned into a pre-modified lentiviral vector (PLVX-EF1A), respectively. Specifically, DLL3 CAR comprises a polypeptide from the N-terminus to the C-terminus: a CD8α signal peptide (CD8α SP, SEQ ID NO: 5), an antigen binding domain having two anti-DLL3 V_{H}H domains (Anti-DLL3 VHH1-VHH2, SEQ ID NOs: 6-7), a CD8α hinge domain (CD8α Hinge, SEQ ID NO: 8), a CD8α transmembrane domain (CD8α TM, SEQ ID NO: 9), a CD137 co-stimulatory signaling domain (4-1BB, SEQ ID NO: 10), and CD3ζ primary intracellular signaling domain (CD3), SEQ ID NO: 11). The YAP comprises a polypeptide with an amino acid sequence of SEQ ID NO: 1 (wildtype YAP, abbreviated as Y wt) or SEQ ID NO: 2 (mutant YAP, abbreviated as Y mut). The WWTR1 comprises a polypeptide with an amino acid sequence of SEQ ID NO: 3 (wildtype WWTR1, abbreviated as T wt) or SEQ ID NO: 4 (mutant WWTR1, abbreviated as T mut). Nucleotides encoding Y wt, Y mut, T wt or T mut were connected with DLL3 CAR by a 2A linker (e.g., T2A or P2A) having an amino acid sequence of SEQ ID NO: 17 or SEQ ID NO: 18.

T cells were isolated from healthy donor PBMCs (HemaCare) using pan T cell isolation kit (Miltenyi Biotec, 130096535). Isolated T cells were cultured under AIMV (Gibco, 31035025) medium with 5% FBS (Gibco, 10099141) and further activated by CD3/CD28 activation beads (Miltenyi Biotec, 130091441) at a ratio of 2:1 in the 37° C., 5% CO₂ incubator. 48 h or 72 h after initial activation, T cells were transduced with lentivirus vectors encoding a DLL3 CAR and/or armors described above at proper multiplicity of infection (MOI) in the presence of 8 μg/mL polybrene (SIGMA-ALDRICH, H9268-10G). Additional IL-2 was supplemented to reach a final concentration of 300 IU/mL. Fresh medium was replaced 24 h post lentiviral infection. Infected T cells were maintained under AIMV medium with 5% FBS and 300 IU/mL IL-2 at a cell density between 5E+05 to 1E+06 cells/mL. UnT refers to T cells un-transduced with CAR.

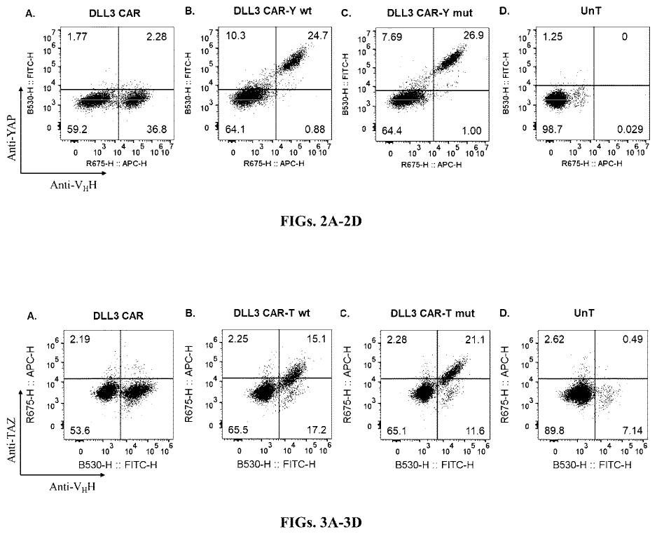

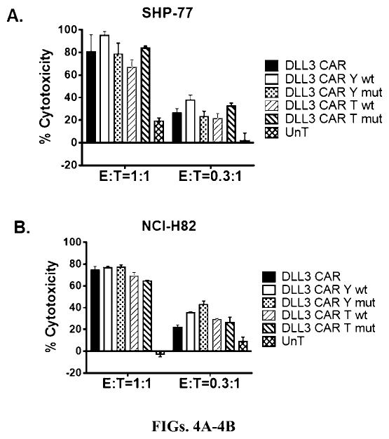

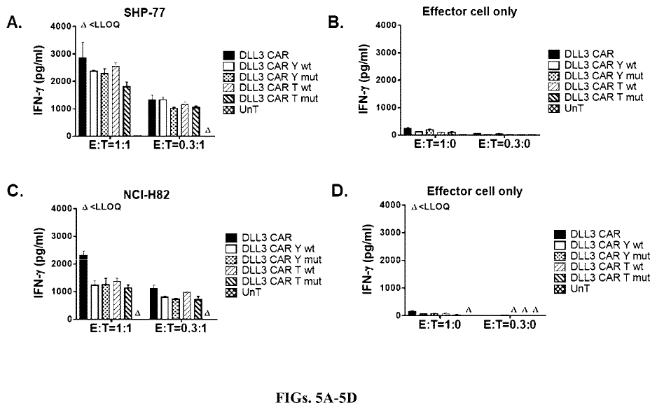

DLL3 CAR expression on T cells described above was determined at 4 days post infection with lentivirus by a rabbit Anti-VHH antibody (Genscript) or Human DLL3 Protein via flow cytometry (BD FACsCelesta). CAR positive rate and geometric mean expression (mean fluorescent intensity, MFI) was further analyzed by FlowJo_v10.8.1. YAP and WWTR1 expression were measured by intracellular staining with antibodies specifically recognizing YAP or WWTR1 (CST, 14729S and 83669S for YAP and WWTR1, respectively). As shown in FIGS. 2A-2D, DLL3 CARs and wildtype or mutant YAP were successfully expressed in each CAR T groups. The CAR and YAP double positive rate was 24.7% (FIG. 2B) in DLL3 CAR-Y wt cells and 26.9% (FIG. 2C) in DLL3 CAR-Y mut cells, respectively. Similarly, as shown in FIGS. 3A-3D, DLL3 CARs and wildtype or mutant WWTR1 were successfully expressed in each CAR T groups. The CAR and WWTR1 double positive rate were 15.1% (FIG. 3B) in DLL3 CAR-T wt cells and 21.1% (FIG. 3C) in DLL3 CAR-T mut cells, respectively. Of note, in line with previous findings, the protein stability of WWTR1 was substantially improved upon mutating LATS phosphorylation sites (see FIGS. 3B-3C). The LATS phosphorylation mutation sites of YAP were S61A, S109A, S127A, S164A, and/or S381A, and the LATS phosphorylation mutation sites of WWTR1 were S66A, S89A, S117A, and/or S311A, respectively.

### Example 3. Cytotoxicity of CAR T Cells

To assay the cytotoxicity of CAR T cells transfected with lentiviruses described in the Example 2 towards tumor associated antigen (TAA) expressing tumor cells, an LDH based cytotoxicity assay were used. Briefly, T cells were cultured with different tumor cell lines (DLL3-positive SHP-77 and NCI-H82 cells) at an ET ratio of 0.3:1 and 1:1 for 24 hours, respectively. The release of LDH from the dead and dying cells in the supernatant was detected by PHERAStar microplate reader according to manufacturer's protocol (Roche, 11644793001). Baseline LDH released from the target cell in the absence of effector cells and effector cell in the absence of target cells were subtracted from the total LDH amount. The target maximum release was determined by adding Triton-X100 at a final concentration of 1% to target cells in the absence of effector cells at the time when the cytotoxicity assays were initiated. Target minimum release was determined using supernatant from target cells in the absence of effector cells. The specific cytotoxicity was calculated by the following formula: % Target cell lysis=100×[(OD CAR T cell+Target cell)−(OD CAR T cell)−(OD Target Minimum release)+(OD Buffer background)]/(OD Target Maximum release−OD Target Minimum release).

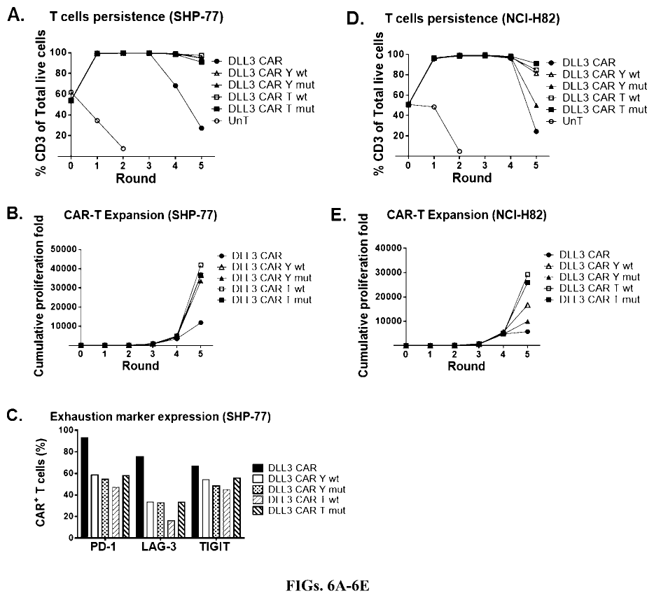

As shown in FIG. 4A, in contrast to little cytotoxicity of UnT against tumor cells, specific cytotoxicity was observed in CAR T cell groups against SHP-77 cells. Conventional DLL3 CAR T cells exhibited approximately 80% and 30% cytotoxicity at an E:T ratio of 1:1 and 0.3:1, respectively (FIG. 4A); and YAP and WWTR1 overexpressing DLL3 CAR T cells induced comparable cytotoxicity (FIG. 4A). The cytotoxicity was additionally determined against NCI-H82 cells, a cell line is more resistant to CAR T mediated cytolysis comparing to SHP-77, due to decreased DLL3 expression and deregulated cell death pathways. Interestingly, while comparable cytotoxicity around 70% was observed at an E:T ratio of 1:1, cytotoxicity of YAP or WWTR1 overexpressing CAR T cells was found to be up-regulated by 1.2-2 folds at an E:T ratio of 0.3:1 (FIG. 4B).

### Example 4. IFN-γ Released by CAR T Cells

To further analyze the effects of CAR T cells against tumor cells, interferon-γ release was analyzed in the culture supernatant using the HTRF human IFN gamma kit (Cisbio, 62HIFNGPEH).

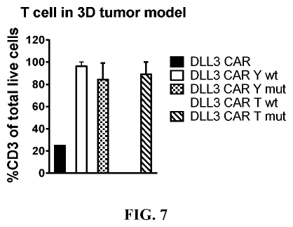

Cytokine analysis from the SHP-77 co-culture supernatant showed similar IFN-γ release in conventional DLL3 CAR T cells and YAP overexpressing DLL3 CAR T cells (FIG. 5A). Notably, limited baseline cytokine released by CAR T cells suggested the low tonic activity (FIG. 5B). Cytokine analysis from the SHP-77 co-culture supernatant showed reduced INF-γ release by WWTR1 overexpressing DLL3 CAR T cells comparing to conventional DLL3 CAR T cells at an E:T ratio of 1:1; however, only slight decrease of INF-γ release was observed at an E: T ratio of 0.3:1 (FIG. 5C). Again, minimal baseline cytokine was released by CAR T cells alone (FIG. 5D), confirming the little tonic activity of CAR T cells.

### Example 5. Persistent Cytotoxicity Assayed by Repetitive DLL3-Mediated Stimulations

Persistence of CAR T cells with different vectors described above were evaluated in a tumor re-challenge assay mediated by SHP-77 and NCI-H82 SCLC tumor cells, respectively. In brief, 7.5E+04 CAR positive T cells were co-cultured with 3E+05 SHP-77 or NCI-H82 cells in a 24 well plate, respectively. Three or four days later, cells were harvested to determine the relative ratio of viable T cell and tumor cell, CAR positive T cells were quantified and re-plated with fresh SHP-77 or NCI-H82 cells at an E:T ratio of 1:4 for the next round, respectively. The harvesting and re-plating processes were repeated following the ET ratio of 1:4 until clear persistence difference was observed.

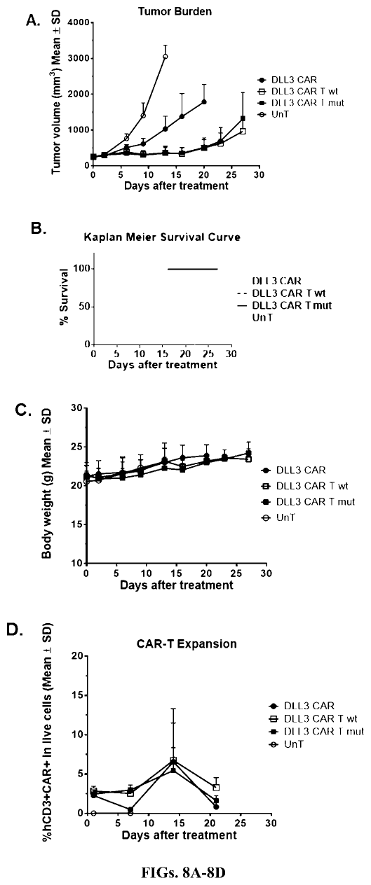

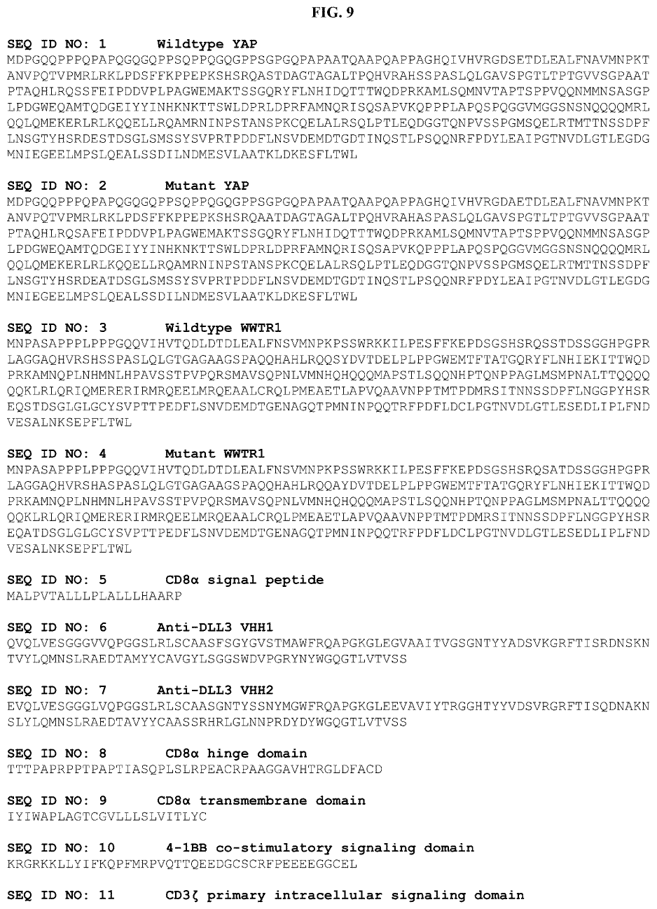

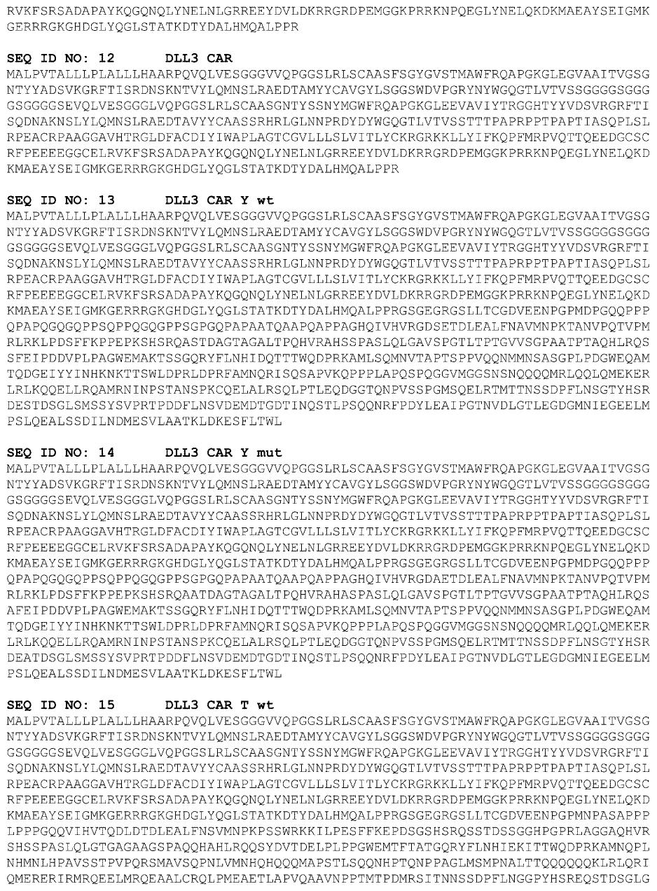

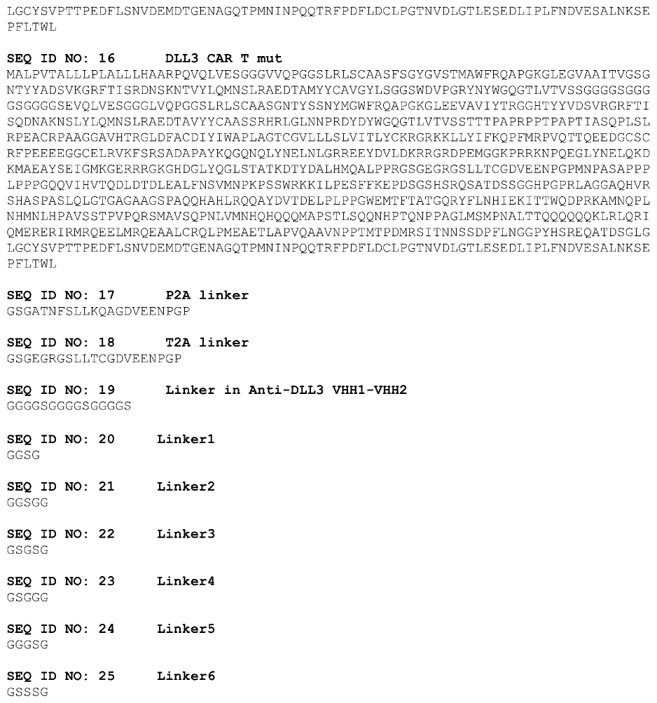

As shown in FIGS. 6A-6C, the significant improvements in persistence were observed in YAP or WWTR1 overexpressing CAR T cells in comparison to conventional DLL3 CAR T cells, irrespective of wildtype or mutant YAP and WWTR1. The T persistence in response to repetitive tumor associated antigen challenge mediated by SHP-77 tumor cells was substantially enhanced with at least a one-round advantage by simultaneously overexpression of YAP or WWTR1 (FIG. 6A). Further, the proliferation of YAP or WWTR1 overexpressing DLL3 CAR T cells was significantly enhanced in comparison to conventional DLL3 CAR T cells by about 3-4-folds for CAR-positive T cells (FIG. 6B). Analysis of CAR T cell exhaustion by measuring the PD-1, LAG-3 and TIGIT expression further confirmed that overexpressing of YAP or WWTR1 permitted less exhaustive differentiation upon tumor associated antigen (TAA) engagement (FIG. 6C).

The analysis of T cell persistence and proliferation in response to NCI-H82-mediated repetitive stimulation confirmed the superiority of YAP or WWTR1 overexpressing CAR T cells in comparison to conventional DLL3 CAR T cells. While the percentage of CD3 population in the conventional DLL3 CAR T cells dropped to around 20% after 5 rounds of tumor challenge, the percentage of CD3 population was significantly higher in the YAP or WWTR1 overexpressing CAR T cells. Specifically, DLL3 Y wt, DLL3 T wt and DLL3 CAR T mut positive T cells showed around 80% T cells remaining in the co-culture well (FIG. 6D), after 5 rounds of tumor challenge. Further, in comparison to conventional DLL3 CAR T cells, the CAR positive T proliferation was increased by about 2.9, 1.7, 5.1 and 4.5-folds for DLL3 CAR Y wt, DLL3 CAR Y mut, DLL3 CAR T wt and DLL3 CAR T mut positive T cells, respectively (FIG. 6E).

### Example 6. Migration and Persistence of CAR T Cells in a 3D-Tumor Model

To further investigate the penetration and persistence of CAR T cells in a solid tumor-mimicking condition, an ex vivo 3D tumor model was developed. In brief, 5E+03 SHP-77 cells were mixed with 80 μL of liquid Matrigel (Corning, 354230), the tumor cell containing Matrigel was consolidated after incubating for half an hour at 37° C. The 3D tumor spheroids were cultured for 5-7 days by topping up 150 μL additional 1640 culture medium with 10% heat-inactivated FBS. The tumor cell number from the counting wells was determined to determine the cell number. DLL3 CAR T cells, and YAP or WWTR1 armored DLL3 CAR T cells as described above were added at an E:T ratio of 1:5 to experimental wells in 150 μL AIMV culture medium. The supernatant and un-infiltrated T cells were removed 48 hours later by refreshing 150 μL of AIMV medium. The T cell percentage within the 3D Matrigel was analyzed (by dissolving the 3D Matrigel) 5-7 days post co-culture to determine CAR T cell penetration and ex vivo proliferation. As shown in FIG. 7, while 20% T cells were found for DLL3 CAR T cells, the T cell percentage was found to be around 80-90% for both wildtype and mutant versions of YAP and WWTR1 overexpressing DLL3 CAR T cells, confirming the superiority of YAP or WWTR1 in inducing T cell mediated anti-tumor immune responses.

### Example 7. In Vivo Efficacy of WWTR1 Overexpressing DLL3 CAR T Cells in Comparison to Conventional DLL3 CAR T Cells

To further study the anti-tumor activities of WWTR1 overexpressing CAR T cells in vivo, the efficacy and pharmacokinetics of DLL3 CAR T cells and wildtype or mutant WWTR1 armored DLL3 CAR T cells against an xenograft model derived from SCLC tumor cell line NCI-H82 were determined. NCG mice (NOD-PrkdcCd5I12rgCd/NjuCrl) were subcutaneously injected with NCI-H82 cells. A single dose of un-transduced T cells (8.2E+06), T cells overexpressing conventional DLL3 CAR, T cells overexpressing DLL3 CAR T wt, or T cells overexpressing DLL3 CAR T mut (1.5E+06) were administered intravenously to tumor engrafted mice 10 days after tumor inoculation, respectively (FIGS. 8A-8D). Tumor length (L) and width (W) were measured by caliper every 3-4 days after CAR T cells treatment. Tumor volume was estimated using the following formula: V=(W2×L)/2. Fourteen days after treatment, the NCG mice treated with conventional DLL3 CAR T cells and DLL3 CAR T wt T cells and DLL3 CAR T mut T cells all showed reduced tumor burden comparing with the un-transduced T cells-treated group (FIG. 8A). Tumor regression of DLL3 CAR T wt and DLL3 CAR T mut T cells was more pronounced than the conventional DLL3 CAR T cells treated group. Kaplan-Meier survival curve analysis shown in FIG. 8B suggested that DLL3 CAR T cells treated mice died starting from day 16 (1/5) and were all (4/4) sacrificed 20 days post infusion due to oversized tumor. In contrast, all the experimental mice (5/5 per group) treated with DLL3 CAR T wt and DLL3 CAR T mut T cells survived till the end of observation day 27. The data highlighted the superiority of WWTR1 overexpressing DLL3 CAR T cells in tumor regression and protection of mice from disease progression. No significant weight loss was observed from NCG mice receiving either conventional DLL3 CAR T cells or DLL3 CAR T wt and DLL3 CAR T mut T cells, suggesting both conventional DLL3 CAR T cells and wildtype or mutant WWTR1 overexpressing DLL3 CAR T cells were well tolerated by NCG mice (FIG. 8C).

The expansion and persistence of CAR T cells in vivo are also considered as critical predictors of durable clinical tumor regression in patients with cancer. To understand the basic kinetics of infused CAR T cells, the percentage of CAR positive T cells in peripheral blood of NCG mice was assessed using flow cytometry. As shown in FIG. 8D, while the expansion of conventional DLL3 CAR T cells dropped dramatically to around 0.49%, the CAR positive rates of DLL3 CAR T wt and DLL3 CAR T mut T cells were about 2.5-2.9% in peripheral blood of NCG mice 7 days post treatment. Similar CAR positive rate (5.5%-6.8%) were observed in the peripheral blood of NCG mice treated with different version of DLL3 CAR cells 14 days post treatment (FIG. 8D). This data suggests early expansion of WWTR1 overexpressing DLL3 CAR T cells and enhanced anti-tumor activity of WWTR1 overexpressing DLL3 CAR T cells might result from improved tumor penetration and/or proliferation. In line with minimal weight loss, no TAA-independent CAR T expansion was observed during the study as reflected by constrained proliferation at day 21 (FIG. 8D).

### Other Embodiments

It is to be understood that while the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

What is claimed is:

1. An engineered cell having an enhanced TEAD (transcriptional enhanced associate domain) protein transcriptional activity.

2. The engineered cell of claim 1, wherein the transcriptional activities of a TEAD coactivator are increased.

3. The engineered cell of claim 2, wherein the TEAD coactivator is selected from the group consisting of Yes-associated protein (YAP), WW domain-containing transcription regulator protein 1 (WWTR1), Vestigial like family member 4 (VgLL) and p160 coactivators (SRC-1, GRIP1/TIF2, and pCIP/RAC3/ACTR/AIB1/TRAM1).

4. The engineered cell of claim 3, wherein TEAD coactivator is a YAP peptide or a WWTR1 peptide.

5. The engineered cell of claim 4, wherein the engineered cell overexpresses a YAP peptide or a WWTR1 peptide.

6. The engineered cell of any one of claims 1-5, wherein activities of an upstream regulator of the Hippo pathway are modulated, wherein the upstream regulator is selected from the group consisting of mammalian STE20-like protein kinases 1 and 2, and large tumor suppressor homologs 1 and 2 (LATS1/2), human Mps one binder 1A or 1B (MOB1A/B), human Savaldor (Sav1).

7. An engineered cell overexpressing a YAP peptide or a WWTR1 peptide.

8. The engineered cell of claim 7, wherein the engineered cell further expresses an engineered receptor.

9. An engineered cell comprising:
(a). an engineered receptor; and
(b). an exogenous YAP peptide or an exogenous WWTR1 peptide.

10. The engineered cell of any one of claims 4-9, wherein the YAP peptide is a wildtype YAP peptide or a mutant YAP peptide; and the WWTR1 peptide is a wildtype WWTR1 peptide or a mutant WWTR1 peptide.

11. The engineered cell of any one of claims 4-10, wherein the YAP peptide comprises an amino acid sequence of SEQ ID NO: 1 or SEQ ID NO: 2, or that is at least 95%, 99%, or 100% identical to the amino acid sequence set forth in SEQ ID NO: 1 or SEQ ID NO: 2.

12. The engineered cell of any one of claims 4-10, wherein the WWTR1 peptide comprises an amino acid sequence of SEQ ID NO: 3 or SEQ ID NO: 4, or that is at least 95%, 99%, or 100% identical to the amino acid sequence set forth in SEQ ID NO: 3 or SEQ ID NO: 4.

13. The engineered cell of any one of claims 8-12, wherein the engineered receptor is selected from the group consisting of an engineered T cell receptor (TCR), a chimeric antigen receptor (CAR), a T cell antigen coupler (TAC) or a portion thereof.

14. The engineered cell of claim 13, wherein the engineered receptor is a CAR.

15. The engineered cell of claim 14, wherein the CAR comprises an extracellular antigen binding domain that specifically binds to an antigen.

16. The engineered cell of claim 15, wherein the antigen is a tumor antigen selected from the group consisting of BCMA, CLL1, CD4, GPC3, GPRC5D, GU2CYC, CD19, MUC16, MUC1, CAIX, CEA, CD8, CD7, CD10, CD20, CD22, CD30, CD33, CD34, CD38, CD41, CD44, CD49f, CD56, CD74, CD133, CD138, EGP-2, EGP-40, EpCAM, ERBB2, ERBB3, ERBB4, FBP, fetal acetylcholine receptor, folate receptor-α, GD2, GD3, HER-2, hTERT, IL-13R-α2, κ-light chain, KDR, LeY, L1 cell adhesion molecule, MAGE-A1, mesothelin, MAGEA3, p53, MART1, GP100, proteinase-3 (PR3), tyrosinase, survivin, hTERT, EphA2, NY-ESO-1, h5T4, PSCA, PSMA, ROR1, TAG-72, VEGF-R2, WT-1, CD123, CD44V6, NKCS1, IGF1R, EGFR, EGFR-VIII, Claudin 18.2, Claudin 6, NKG2D, Delta-like 3 (DLL3), CD70, CS-1, c-Met, Glycolipid F77, PD-L1, and PD-L2.

17. The engineered cell of claim 16, wherein the antigen is DLL3.

18. The engineered cell of claim 17, wherein the CAR comprises an amino acid sequence of SEQ ID NO: 12, or an amino acid sequence that is at least 95%, 99%, or 100% identical to the amino acid sequence of SEQ ID NO: 12.

19. The engineered cell of claim 17 or claim 18, comprising a polypeptide having an amino acid sequence set forth in any one of SEQ ID NOs: 13-16, or an amino acid sequence that is at least 95%, 99%, or 100% identical to the amino acid sequence set forth in any one of SEQ ID NOs: 13-16.

20. The engineered cell of any one of claims 4-19, wherein the overexpression of the YAP peptide or the WWTR1 peptide is controlled by a non-native regulatory element.

21. A nucleic acid, comprising one or more nucleic acid sequences encoding:
(a). an engineered receptor; and/or
(b). a YAP peptide or WWTR1 peptide,
wherein the YAP peptide or WWTR1 peptide is overexpressed.

22. The nucleic acid of claim 21, wherein the nucleic acid sequence comprising one, two, three, four, five or more copies of the coding sequence of a YAP peptide or a WWTR1 peptide; or wherein the overexpression of the YAP peptide or the WWTR1 peptide is controlled by a non-native regulatory element.

23. The nucleic acid of claim 21 or claim 22, wherein the YAP peptide comprises an amino acid sequence of SEQ ID NO: 1 or SEQ ID NO: 2, or an amino acid sequence that is at least 95%, 99%, or 100% identical to the amino acid sequence set forth in SEQ ID NO: 1 or SEQ ID NO: 2.

24. The nucleic acid of claim 21 or claim 22, wherein the WWTR1 peptide comprises an amino acid sequence of SEQ ID NO: 3 or SEQ ID NO: 4, or an amino acid sequence that is at least 95%, 99%, or 100% identical to the amino acid sequence set forth in SEQ ID NO: 3 or SEQ ID NO: 4.

25. The nucleic acid of any one of claims 21-24, wherein the nucleic acid sequence encoding the engineered receptor and the nucleic acid sequence encoding the YAP peptide or the WWTR1 peptide are separated by a third nucleic acid sequence encoding a cleavable linker.

26. The nucleic acid of claim 25, wherein the cleavable linker comprises an amino acid sequence set forth in SEQ ID NO: 17 or SEQ ID NO: 18, or a functional variant having at least about 90% sequence identity to the amino acid sequence of SEQ ID NO: 17 or SEQ ID NO: 18.

27. The nucleic acid of any one of claims 21-26, wherein the engineered receptor is selected from the group consisting of an engineered T cell receptor (TCR), a chimeric antigen receptor (CAR), a T cell antigen coupler (TAC) or a portion thereof.

28. The nucleic acid of claim 27, wherein the engineered receptor is a CAR.

29. The nucleic acid of claim 28, wherein the CAR comprises an amino acid sequence of SEQ ID NO: 12, or an amino acid sequence that is at least 95%, 99%, or 100% identical to the amino acid sequence of SEQ ID NO: 12.

30. The nucleic acid of any one of claims 21-29, wherein the nucleic acid sequence encodes an amino acid sequence set forth in any one of SEQ ID NOs: 13-16, or an amino acid sequence that is at least 95%, 99%, or 100% identical to an amino acid sequence set forth in any one of SEQ ID NOs: 13-16.

31. A vector comprising the nucleic acid of any one of claims 21-30.

32. An engineered cell comprising the nucleic acid of any one of claims 21-30, or the vector of claim 31.

33. The engineered cell of any one of claims 7-20 and 32, wherein the engineered cell is an immune cell.

34. The engineered cell of claim 33, wherein the immune cell is selected from a group consisting of T cell, αβT cell, γδT cell, NK cell, NKT, peripheral blood mononuclear cell (PBMC), hematopoietic stem cell, pluripotent stem cell, an embryonic stem cell, and a combination thereof.

35. The engineered cell of claim 34, wherein the immune cell is a T cell.

36. A pharmaceutical composition comprising the engineered cell of any one of claims 7-20 and 32-35, and a pharmaceutically acceptable carrier.

37. A method for producing engineered cell of any one of claims 7-20 and 32-35, the method comprising introducing the vector of claim 31 into a cell.

38. A method of treating a disease or disorder in a subject, the method comprising administering to the subject, an effective amount of the engineered cell of any one of claims 7-20 and 32-35, or the pharmaceutical composition of claim 36.

39. The method of claim 38, wherein the disease or disorder is cancer, autoimmune disease, or infection.

40. The method of claim 38, wherein the disease or disorder is solid tumor.

41. The method of any one of claims 38-40, wherein the disease or disorder is selected from a group consisting of small cell lung cancer (SCLC), large cell neuroendocrine cancer (LCNC), neuroendocrine prostate cancer (NEPC), pancreatic neuroendocrine tumor (PNET) and gastrointestinal neuroendocrine cancers.


SEQUENCE LISTING (25 sequences)

SEQ ID NO: 1 - protein, 504 aa, synthetic construct
  source 1..504: note = Wildtype YAP
MDPGQQPPPQPAPQGQGQPPSQPPQGQGPPSGPGQPAPAATQAAPQAPPAGHQIVHVRGDSETDLEALFNAVMNPKTANVPQTVPMRLRKLPDSFFKPPEPKSHSRQASTDAGTAGALTPQHVRAHSSPASLQLGAVSPGTLTPTGVVSGPAATPTAQHLRQSSFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQAMRNINPSTANSPKCQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 2 - protein, 504 aa, synthetic construct
  source 1..504: note = Mutant YAP
MDPGQQPPPQPAPQGQGQPPSQPPQGQGPPSGPGQPAPAATQAAPQAPPAGHQIVHVRGDAETDLEALFNAVMNPKTANVPQTVPMRLRKLPDSFFKPPEPKSHSRQAATDAGTAGALTPQHVRAHASPASLQLGAVSPGTLTPTGVVSGPAATPTAQHLRQSAFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQAMRNINPSTANSPKCQELALRSQLPTLEQDGGTQNPVSSPGMSQELRTMTTNSSDPFLNSGTYHSRDEATDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 3 - protein, 400 aa, synthetic construct
  source 1..400: note = Wildtype WWTR1
MNPASAPPPLPPPGQQVIHVTQDLDTDLEALFNSVMNPKPSSWRKKILPESFFKEPDSGSHSRQSSTDSSGGHPGPRLAGGAQHVRSHSSPASLQLGTGAGAAGSPAQQHAHLRQQSYDVTDELPLPPGWEMTFTATGQRYFLNHIEKITTWQDPRKAMNQPLNHMNLHPAVSSTPVPQRSMAVSQPNLVMNHQHQQQMAPSTLSQQNHPTQNPPAGLMSMPNALTTQQQQQQKLRLQRIQMERERIRMRQEELMRQEAALCRQLPMEAETLAPVQAAVNPPTMTPDMRSITNNSSDPFLNGGPYHSREQSTDSGLGLGCYSVPTTPEDFLSNVDEMDTGENAGQTPMNINPQQTRFPDFLDCLPGTNVDLGTLESEDLIPLFNDVESALNKSEPFLTWL

SEQ ID NO: 4 - protein, 400 aa, synthetic construct
  source 1..400: note = Mutant WWTR1
MNPASAPPPLPPPGQQVIHVTQDLDTDLEALFNSVMNPKPSSWRKKILPESFFKEPDSGSHSRQSATDSSGGHPGPRLAGGAQHVRSHASPASLQLGTGAGAAGSPAQQHAHLRQQAYDVTDELPLPPGWEMTFTATGQRYFLNHIEKITTWQDPRKAMNQPLNHMNLHPAVSSTPVPQRSMAVSQPNLVMNHQHQQQMAPSTLSQQNHPTQNPPAGLMSMPNALTTQQQQQQKLRLQRIQMERERIRMRQEELMRQEAALCRQLPMEAETLAPVQAAVNPPTMTPDMRSITNNSSDPFLNGGPYHSREQATDSGLGLGCYSVPTTPEDFLSNVDEMDTGENAGQTPMNINPQQTRFPDFLDCLPGTNVDLGTLESEDLIPLFNDVESALNKSEPFLTWL

SEQ ID NO: 5 - protein, 21 aa, synthetic construct
  source 1..21: note = CD8a signal peptide
MALPVTALLLPLALLLHAARP

SEQ ID NO: 6 - protein, 125 aa, synthetic construct
  source 1..125: note = Anti-DLL3 VHH1
QVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSS

SEQ ID NO: 7 - protein, 125 aa, synthetic construct
  source 1..125: note = Anti-DLL3 VHH2
EVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSS

SEQ ID NO: 8 - protein, 45 aa, synthetic construct
  source 1..45: note = CD8a hinge domain
TTTPAPRPPTPAPTIASQPLSLRPEACRPAAGGAVHTRGLDFACD

SEQ ID NO: 9 - protein, 24 aa, synthetic construct
  source 1..24: note = CD8a transmembrane domain
IYIWAPLAGTCGVLLLSLVITLYC

SEQ ID NO: 10 - protein, 42 aa, synthetic construct
  source 1..42: note = 4-1BB co-stimulatory signaling domain
KRGRKKLLYIFKQPFMRPVQTTQEEDGCSCRFPEEEEGGCEL

SEQ ID NO: 11 - protein, 112 aa, synthetic construct
  source 1..112: note = CD3z primary intracellular signaling domain
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 509 aa, synthetic construct
  source 1..509: note = DLL3 CAR
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 1034 aa, synthetic construct
  source 1..1034: note = DLL3 CAR Y wt
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMDPGQQPPPQPAPQGQGQPPSQPPQGQGPPSGPGQPAPAATQAAPQAPPAGHQIVHVRGDSETDLEALFNAVMNPKTANVPQTVPMRLRKLPDSFFKPPEPKSHSRQASTDAGTAGALTPQHVRAHSSPASLQLGAVSPGTLTPTGVVSGPAATPTAQHLRQSSFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQAMRNINPSTANSPKCQELALRSQLPTLEQDGGTQNPVSSPGMSQELRTMTTNSSDPFLNSGTYHSRDESTDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 14 - protein, 1034 aa, synthetic construct
  source 1..1034: note = DLL3 CAR Y mut
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMDPGQQPPPQPAPQGQGQPPSQPPQGQGPPSGPGQPAPAATQAAPQAPPAGHQIVHVRGDAETDLEALFNAVMNPKTANVPQTVPMRLRKLPDSFFKPPEPKSHSRQAATDAGTAGALTPQHVRAHASPASLQLGAVSPGTLTPTGVVSGPAATPTAQHLRQSAFEIPDDVPLPAGWEMAKTSSGQRYFLNHIDQTTTWQDPRKAMLSQMNVTAPTSPPVQQNMMNSASGPLPDGWEQAMTQDGEIYYINHKNKTTSWLDPRLDPRFAMNQRISQSAPVKQPPPLAPQSPQGGVMGGSNSNQQQQMRLQQLQMEKERLRLKQQELLRQAMRNINPSTANSPKCQELALRSQLPTLEQDGGTQNPVSSPGMSQELRTMTTNSSDPFLNSGTYHSRDEATDSGLSMSSYSVPRTPDDFLNSVDEMDTGDTINQSTLPSQQNRFPDYLEAIPGTNVDLGTLEGDGMNIEGEELMPSLQEALSSDILNDMESVLAATKLDKESFLTWL

SEQ ID NO: 15 - protein, 930 aa, synthetic construct
  source 1..930: note = DLL3 CAR T wt
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMNPASAPPPLPPPGQQVIHVTQDLDTDLEALFNSVMNPKPSSWRKKILPESFFKEPDSGSHSRQSSTDSSGGHPGPRLAGGAQHVRSHSSPASLQLGTGAGAAGSPAQQHAHLRQQSYDVTDELPLPPGWEMTFTATGQRYFLNHIEKITTWQDPRKAMNQPLNHMNLHPAVSSTPVPQRSMAVSQPNLVMNHQHQQQMAPSTLSQQNHPTQNPPAGLMSMPNALTTQQQQQQKLRLQRIQMERERIRMRQEELMRQEAALCRQLPMEAETLAPVQAAVNPPTMTPDMRSITNNSSDPFLNGGPYHSREQSTDSGLGLGCYSVPTTPEDFLSNVDEMDTGENAGQTPMNINPQQTRFPDFLDCLPGTNVDLGTLESEDLIPLFNDVESALNKSEPFLTWL

SEQ ID NO: 16 - protein, 930 aa, synthetic construct
  source 1..930: note = DLL3 CAR T mut
MALPVTALLLPLALLLHAARPQVQLVESGGGVVQPGGSLRLSCAASFSGYGVSTMAWFRQAPGKGLEGVAAITVGSGNTYYADSVKGRFTISRDNSKNTVYLQMNSLRAEDTAMYYCAVGYLSGGSWDVPGRYNYWGQGTLVTVSSGGGGSGGGGSGGGGSEVQLVESGGGLVQPGGSLRLSCAASGNTYSSNYMGWFRQAPGKGLEEVAVIYTRGGHTYYVDSVRGRFTISQDNAKNSLYLQMNSLRAEDTAVYYCAASSRHRLGLNNPRDYDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMNPASAPPPLPPPGQQVIHVTQDLDTDLEALFNSVMNPKPSSWRKKILPESFFKEPDSGSHSRQSATDSSGGHPGPRLAGGAQHVRSHASPASLQLGTGAGAAGSPAQQHAHLRQQAYDVTDELPLPPGWEMTFTATGQRYFLNHIEKITTWQDPRKAMNQPLNHMNLHPAVSSTPVPQRSMAVSQPNLVMNHQHQQQMAPSTLSQQNHPTQNPPAGLMSMPNALTTQQQQQQKLRLQRIQMERERIRMRQEELMRQEAALCRQLPMEAETLAPVQAAVNPPTMTPDMRSITNNSSDPFLNGGPYHSREQATDSGLGLGCYSVPTTPEDFLSNVDEMDTGENAGQTPMNINPQQTRFPDFLDCLPGTNVDLGTLESEDLIPLFNDVESALNKSEPFLTWL

SEQ ID NO: 17 - protein, 22 aa, synthetic construct
  source 1..22: note = P2A linker
GSGATNFSLLKQAGDVEENPGP

SEQ ID NO: 18 - protein, 21 aa, synthetic construct
  source 1..21: note = T2A linker
GSGEGRGSLLTCGDVEENPGP

SEQ ID NO: 19 - protein, 15 aa, synthetic construct
  source 1..15: note = Linker in Anti-DLL3 VHH1-VHH2
GGGGSGGGGSGGGGS

SEQ ID NO: 20 - protein, 4 aa, synthetic construct
  source 1..4: note = Linker1
GGSG

SEQ ID NO: 21 - protein, 5 aa, synthetic construct
  source 1..5: note = Linker2
GGSGG

SEQ ID NO: 22 - protein, 5 aa, synthetic construct
  source 1..5: note = Linker3
GSGSG

SEQ ID NO: 23 - protein, 5 aa, synthetic construct
  source 1..5: note = Linker4
GSGGG

SEQ ID NO: 24 - protein, 5 aa, synthetic construct
  source 1..5: note = Linker5
GGGSG

SEQ ID NO: 25 - protein, 5 aa, synthetic construct
  source 1..5: note = Linker6
GSSSG